(19) United States
(12) Patent Application Publication
(10) Pub. No.: US 2025/0299820 A1
(43) Pub. Date: Sep. 25, 2025

(54) MEDICAL INFORMATION PROCESSING APPARATUS, MEDICAL INFORMATION PROCESSING METHOD, AND STORAGE MEDIUM

(71) Applicant: Canon Medical Systems Corporation, Otawara-shi (JP)

(72) Inventors: Junichi TASHIRO, Otawara (JP); Shinya NISHITAKA, Nasushiobara (JP); Yosuke YANAGIDA, Nasushiobara (JP); Yasunari KUTSUNA, Nasushiobara (JP); Kazumasa ARAKITA, Utsunomiya (JP)

(73) Assignee: Canon Medical Systems Corporation, Otawara-shi (JP)

(21) Appl. No.: 19/085,040

(22) Filed: Mar. 20, 2025

(30) Foreign Application Priority Data
Mar. 21, 2024 (JP) 2024-045396
Mar. 19, 2025 (JP) 2025-044573

(51) Int. Cl. G16H 50/20; G16H 10/60; G16H 30/40

(52) U.S. Cl. CPC G16H 50/20; G16H 10/60; G16H 30/40

(57) ABSTRACT

According to one embodiment, a medical information processing apparatus includes processing circuitry. The processing circuitry is configured to acquire examination purpose information regarding an examination purpose including a description regarding a first disease candidate of a patient. The processing circuitry is configured to acquire image interpretation information including the description regarding the first disease candidate from an image interpretation report regarding an image examination performed according to the examination purpose. The processing circuitry is configured to specify an incidental finding described for a second disease candidate different from the first disease candidate from the image interpretation information. The processing circuitry is configured to display a specified result on a display.

### CROSS-REFERENCE TO RELATED APPLICATION(S)

This application is based upon and claims the benefit of priority from the prior Japanese Patent Applications No. 2024-045396, filed Mar. 21, 2024, and No. 2025-044573, filed Mar. 19, 2025, the entire contents of all of which are incorporated herein by reference.

### FIELD

Embodiments described herein relate generally to a medical information processing apparatus, a medical information processing method, and a storage medium.

### BACKGROUND

A medical doctor requests an image examination for an examination purpose such as diagnosing a disease or abnormality assumed for a patient. In the image examination, a medical image of the patient is captured by X-ray computed tomography (CT), magnetic resonance imaging (MRI), or the like. A radiologist displays the captured medical image on a report creation device to perform image interpretation, and creates an image interpretation report describing an abnormality found in the medical image and an assumed disease for the abnormality. Such an image interpretation report may include an incidental finding regarding a disease or abnormality that is not assumed at the time of request.

Meanwhile, in a case of displaying the image interpretation report on a medical information processing apparatus and checking the image interpretation report, the medical doctor focuses on authenticity of the assumption such as whether or not the assumed disease or abnormality is indicated. For this reason, in the image interpretation report, the medical doctor may overlook the incidental finding regarding an unexpected disease or abnormality.

To cope with the case, the following three measures are taken in order not to overlook the incidental finding.

In the first measure, the radiologist assigns an important flag to the incidental finding of the image interpretation report. As a result, it can be expected that the medical doctor also focuses on the important flag and confirms the image interpretation report.

In the second measure, a check system by a third party is established. As a result, it can be expected that the third party checks that the medical doctor has confirmed the incidental finding of the image interpretation report.

In the third countermeasure, unread and read of the image interpretation report by the medical doctor are managed. As a result, it can be expected that the medical doctor is prompted to read the unread image interpretation report.

According to the study of the present inventor, there is room for improvement in the above three measures in the following points. For example, the first measure has a disadvantage such as forgetting to assign the important flag to the incidental finding or assigning the important flag to an unimportant item. This disadvantage is believed to arise due to inability to specify the incidental finding. In the second measure, since a cost of checking by the third party is incurred, the cost increases. In the third measure, even if the medical doctor has read the image interpretation report, he/she does not necessarily focus on the incidental finding.

Therefore, in order not to overlook the incidental finding, it is desirable to specify the incidental finding and give attention to the incidental finding while suppressing an increase in cost.

### BRIEF DESCRIPTION OF DRAWINGS

FIG. 1 is a block diagram illustrating a medical information processing apparatus and a peripheral configuration thereof according to a first embodiment.

FIG. 2 is a block diagram illustrating a configuration of the medical information processing apparatus of FIG. 1.

FIG. 3 is a schematic diagram for describing an example of a flow of image examination in the first embodiment.

FIG. 4 is a schematic diagram illustrating an example of an electronic medical record of FIG. 3.

FIG. 5 is a schematic diagram illustrating an example of an examination order of FIG. 3.

FIG. 6 is a schematic diagram illustrating an example of an image interpretation report of FIG. 3.

FIG. 7 is a flowchart for describing an operation in the first embodiment.

FIG. 8 is a schematic diagram for describing an operation in the first embodiment.

FIG. 9 is a schematic diagram for describing an operation in the first embodiment.

FIG. 10 is a schematic diagram for describing an operation in the first embodiment.

FIG. 11 is a schematic diagram for describing an operation in the first embodiment.

FIG. 12 is a schematic diagram for describing an operation in the first embodiment.

FIG. 13 is a block diagram illustrating a configuration of a medical information processing apparatus according to a second embodiment.

FIG. 14 is a schematic diagram for describing a trained model according to the second embodiment.

FIG. 15 is a block diagram illustrating a medical information processing apparatus according to a modification of the second embodiment and a peripheral configuration thereof.

FIG. 16 is a block diagram illustrating a configuration of a medical information processing apparatus according to a third embodiment.

FIG. 17 is a schematic diagram for describing a trained model according to the third embodiment.

FIG. 18 is a schematic diagram for describing a display example in a graph format in the third embodiment.

FIG. 19 is a schematic diagram for describing another display example in a graph format in the third embodiment.

FIG. 20 is a schematic diagram for describing still another display example in a graph format in the third embodiment.

FIG. 21 is a schematic diagram for describing processing circuitry according to a fourth embodiment.

FIG. 22 is a schematic diagram for describing an example of a flow of endoscopic examination in the fourth embodiment.

FIG. 23 is a schematic diagram for describing an example of an electronic medical record according to the fourth embodiment.

FIG. 24 is a schematic diagram for describing an example of an examination order according to the fourth embodiment.

FIG. 25 is a schematic diagram for describing an example of a pathological report in the fourth embodiment.

FIG. 26 is a schematic diagram for describing an example of an endoscopic report according to the fourth embodiment.

FIG. 27 is a block diagram illustrating a configuration of a medical information processing apparatus according to a fifth embodiment.

FIG. 28 is a schematic diagram for describing processing circuitry according to a fifth embodiment.

### DETAILED DESCRIPTION

In general, according to one embodiment, a medical information processing apparatus includes processing circuitry. The processing circuitry is configured to acquire examination purpose information regarding an examination purpose including a description regarding a first disease candidate of a patient. The processing circuitry is configured to acquire image interpretation information including the description regarding the first disease candidate from an image interpretation report regarding an image examination performed according to the examination purpose. The processing circuitry is configured to specify an incidental finding described for a second disease candidate different from the first disease candidate from the image interpretation information. The processing circuitry is configured to display a specified result on a display.

Hereinafter, a medical information processing apparatus, a medical information processing method, and a medical information processing program according to the present embodiments will be described with reference to the drawings. In the following description, the term “disease” may be any subject that can be read in image examination, and is not limited to a disease having a disease name, and includes an abnormality such as bleeding.

### First Embodiment

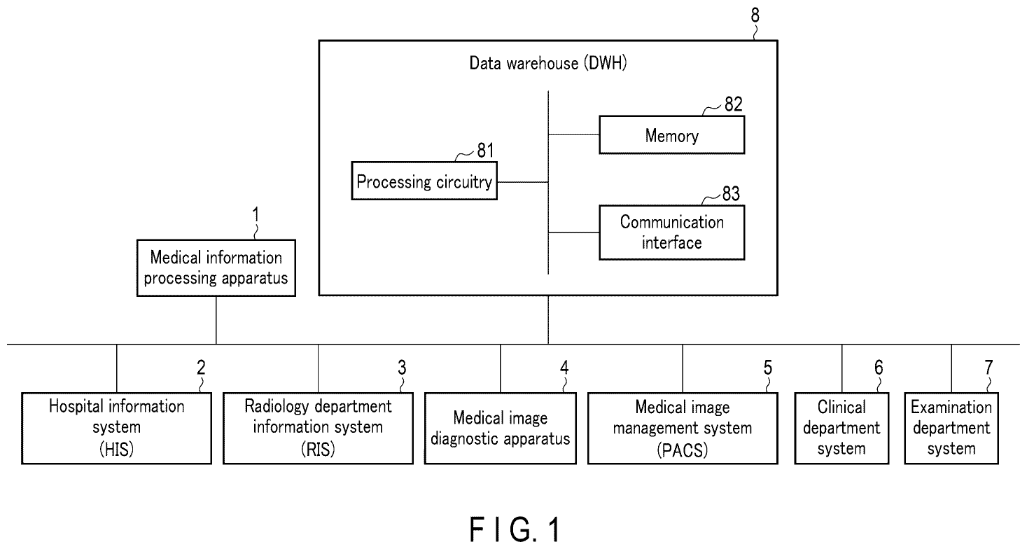

FIG. 1 is a block diagram illustrating a medical information processing apparatus and a peripheral configuration thereof according to a first embodiment. A medical information processing apparatus 1 illustrated in FIG. 1 is an apparatus capable of integrally observing medical information, for example. In the medical information processing apparatus 1, for example, an integrated viewer is mounted. The integrated viewer is an application that presents the medical information to a user in an integrated manner. The integrated viewer may adopt any implementation form such as a web application, a fat client application, or a thin client application. The medical information processing apparatus 1 is communicably connected to a hospital information system (HIS) 2, a radiology department information management system (radiology information system (RIS)) 3, a medical image diagnostic apparatus 4, a medical image management system (picture archiving and communication system (PACS)) 5, a clinical department system 6, an examination department system 7, and a data warehouse (DWH) 8 via an in-hospital network such as a local area network (LAN).

In FIG. 1, the HIS 2 includes, for example, an electronic medical record system that manages information regarding an electronic medical record. The information regarding the electronic medical record includes, for example, patient information and a plurality of pieces of medical information. The patient information is patient-specific information, and includes, for example, a patient ID, patient name, gender, age, and the like.

The plurality of pieces of medical information is associated with the patient ID, and is information obtained by the medical doctor regarding a physical condition, a disease condition, treatment, and the like of the patient in the course of medical examination. Each of the plurality of pieces of medical information individually includes various types of information such as image information, examination history information, electrocardiogram information, vital sign information, drug history information, report information, medical record description information, and nursing record information. Various types of information in the medical information can be distinguished by data types. Further, various types of information included in each of the image information, examination history information, electrocardiogram information, vital sign information, drug history information, report information, medical record description information, and nursing record information can also be similarly distinguished by data types. The image information is, for example, information indicating a location of a medical image acquired by capturing the patient or the like. The image information includes, for example, information indicating a location of a medical image file to be described below generated by the medical image diagnostic apparatus 4 as a result of an examination. The examination history information is, for example, information indicating a history of examination results acquired as a result of performing a specimen examination, a bacterial examination, and the like on the patient. The electrocardiogram information is, for example, information regarding an electrocardiogram waveform measured from the patient. The vital sign information is, for example, basic information related to the patient's life. The vital sign information includes, for example, a pulse rate, a respiratory rate, an oxygen concentration, a body temperature, a blood pressure, a level of consciousness, and the like. The drug history information is, for example, information indicating a history of the amount of a drug administered to the patient. The report information is, for example, information summarized by the radiologist who has interpreted the medical image such as an X-ray image, a CT image, an MRI image, and an ultrasound image, and summarizes a state and a disease of the patient in response to an examination request from the medical doctor in a diagnosis and treatment department. The report information includes, for example, an image interpretation report created by the radiologist with reference to the medical image file stored in the PACS 5. Since the report information is generally stored in the PACS 5, the electronic medical record system can display the report information by reading the report information from the PACS 5.

The medical record description information is, for example, information input to the electronic medical record by the medical doctor or the like. The medical record description information includes, for example, a main complaint, a previous disease, a concurrent disease, an internal medicine, a family history, a physical finding, an examination ID, an examination date, an examination type, a site, the examination purpose, suspicion of diagnosis, and examination requester information (medical doctor name).

The nursing record information is, for example, information input to the electronic medical record by a nurse or the like. The nursing record information includes a nursing record at the time of hospitalization and the like.

In addition, the HIS 2 includes, for example, an order system that manages reservation information, order information, and the like. Note that the HIS 2 may have a configuration in which the electronic medical record system includes the ordering system.

The reservation information includes, for example, information regarding a consultation reservation, an examination reservation, and the like. The information regarding the consultation reservation includes, for example, a consultation date, a consultation time, a reception number, a requesting doctor, a requesting department, and the like. The information regarding the examination reservation includes, for example, an examination date, an examination time, a reception number, and the like. The examination order is, for example, information regarding an order requested by the medical doctor or the like, and includes, for example, order information regarding image examination, specimen examination, physiological inspection, prescription, medication, and the like. In a case where the examination order is order information for requesting image examination, the examination order includes, for example, an order number, the patient information, the examination ID, the examination date, the examination type, an examination site, the examination purpose, the suspicion of diagnosis, the examination requester information, and the like. The order number is a number issued in a case where the examination order is input, and is, for example, an identifier for uniquely specifying the examination order in one hospital. The examination ID is an identifier capable of identifying the examination. The examination type includes an X-ray examination, a computed tomography (CT) examination, a magnetic resonance (MR) examination, a radio isotope (RI) examination, a specimen examination, and the like. The examination site includes, for example, abdomen, brain, chest, and the like. The examination requester information includes a diagnosis and treatment department name, a doctor name in charge, and the like. The information regarding the examination reservation is associated with the order information.

The RIS 3 is a system that manages the examination reservation information regarding radiation examination work. For example, the RIS 3 adds various types of setting information to the examination order information input from the medical doctor in the ordering system included in the HIS 2, accumulates the information, and manages the accumulated information as the examination reservation information. Note that the RIS 3 may add various types of setting information to the examination order information by using an irradiation record in which various types of setting information set in the medical image diagnostic apparatus 4 at the time of past examination is recorded. The RIS 3 transmits the examination order to the medical image diagnostic apparatus 4 in accordance with the examination reservation information. In addition, as a result of implementation of the examination, the RIS 3 transmits examination implementation information generated by the medical image diagnostic apparatus 4 to the electronic medical record system included in the HIS 2.

The medical image diagnostic apparatus 4 is an apparatus that implements the examination by capturing the patient or the like. The medical image diagnostic apparatus 4 includes, for example, an X-ray computed tomography apparatus, an X-ray diagnostic apparatus, a magnetic resonance imaging apparatus, a nuclear medicine diagnostic apparatus, an ultrasonic diagnostic apparatus, and the like. The medical image diagnostic apparatus 4 implements the examination based on, for example, the examination reservation information transmitted from the RIS 3. The medical image diagnostic apparatus 4 generates the examination implementation information and transmits the examination implementation information to the RIS 3.

In addition, the medical image diagnostic apparatus 4 generates medical image data by implementing the examination. Examples of the medical image data include X-ray CT image data, X-ray image data, MRI image data, nuclear medicine image data, and ultrasound image data. The medical image diagnostic apparatus 4 generates the medical image file by converting the generated medical image data into a format conforming to digital imaging and communication in medicine (DICOM) standard, for example. The medical image file is, for example, a file in a format conforming to the DICOM standard. The medical image diagnostic apparatus 4 transmits the generated medical image file to the PACS 5.

The PACS 5 is a system that manages various medical image files. The PACS 5 stores, for example, the medical image file transmitted from the medical image diagnostic apparatus 4. Note that the PACS 5 may store the report information attached to the medical image file or the report information for examinations regarding a plurality of the medical image files.

The clinical department system 6 is a system that manages a clinical department. In a case where a specified result regarding a disease candidate of the patient and the patient information regarding the patient are notified from the medical information processing apparatus 1, the clinical department system 6 accumulates and manages the reported contents.

The examination department system 7 is a system that manages the examination reservation information regarding examination work other than the radiation examination work. For example, in a case of receiving the examination reservation information from the ordering system or the medical information processing apparatus 1, the examination department system 7 accumulates and manages the examination reservation information. In addition, the examination department system 7 transmits the examination reservation information to an examination apparatus. In addition, the examination department system 7 transmits the examination implementation information generated by the examination apparatus to the electronic medical record system included in the HIS 2 as a result of the examination.

The DWH 8 is, for example, a database system that collectively accumulates the medical information (medical big data) generated in medical and nursing related organizations. The DWH 8 is realized by, for example, a general server device. The DWH 8 includes, for example, processing circuitry 81, a memory 82, and a communication interface 83 as illustrated in FIG. 1. The processing circuitry 81, the memory 82, and the communication interface 83 are communicably connected to each other via a bus, for example.

The processing circuitry 81 is a processor that functions as a core of the DWH 8. The processing circuitry 81 executes a program stored in the memory 82 or the like to implement a function corresponding to the program. As this function, for example, a function to collect desired information from the HIS 2, the RIS 3, the medical image diagnostic apparatus 4, the PACS 5, and the like and a function to store the collected information in the memory 82 can be appropriately used. As a result, for example, information regarding the electronic medical record is collected from the HIS 2, the examination order is collected from the RIS 3, the medical image file is collected from the medical image diagnostic apparatus 4 or the PACS 5, and the image interpretation report is collected from the PACS 5.

The memory 82 is a storage device such as a hard disk drive (HDD), a solid state drive (SSD), and an integrated circuit storage device that store various types of information. Furthermore, the memory 82 may be a drive device or the like that reads and writes various types of information from and to a portable storage medium such as a CD-ROM drive, a DVD drive, or a flash memory. The memory 82 stores, for example, a control program and the like for causing the processing circuitry 81 to realize each function such as the function to collect desired information and the function to store the collected information in the memory 82. Note that the program may be stored in a non-transitory storage medium and distributed, for example, and may be read from the non-transitory storage medium and installed in the memory 82.

The communication interface 83 performs data communication with the medical information processing apparatus 1, the HIS 2, the RIS 3, the medical image diagnostic apparatus 4, the PACS 5, the clinical department system 6, and the examination department system 7 connected via the in-hospital network. The standards of communication with the medical information processing apparatus 1, the HIS 2, the RIS 3, the medical image diagnostic apparatus 4, the PACS 5, the clinical department system 6, and the examination department system 7 may be any standards, and examples thereof include Health Level 7 (HL7), DICOM, or both of them.

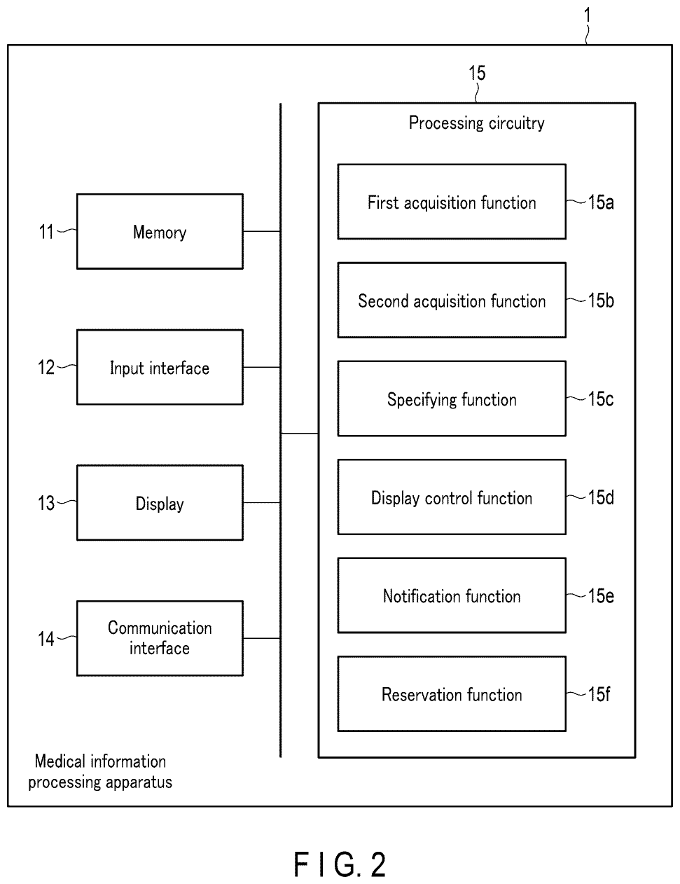

Next, details of the medical information processing apparatus 1 will be described with reference to FIG. 2. FIG. 2 is a block diagram illustrating a configuration of the medical information processing apparatus 1 illustrated in FIG. 1.

The medical information processing apparatus 1 illustrated in FIG. 2 includes a memory 11, an input interface 12, a display 13, a communication interface 14, and processing circuitry 15. The memory 11, the input interface 12, the display 13, the communication interface 14, and the processing circuitry 15 are communicably connected to each other via a bus, for example.

Here, the memory 11 includes memories that record electrical information, such as a read only memory (ROM), a random access memory (RAM), a hardware disk drive (HDD), and an image memory, and peripheral circuits such as a memory controller and a memory interface accompanying these memories. The memory 11 stores, for example, various programs such as a medical information processing program of the medical information processing apparatus 1 and various data such as various tables, data being in processing, and data after processing. Note that the medical information processing program is a program for causing a computer to function as the medical information processing apparatus 1 that executes a medical information processing method. For example, the medical information processing program may be stored and distributed in a non-transitory computer-readable storage medium, read from the storage medium, and installed in the memory 11.

The input interface 12 is realized by a trackball, a switch button, a mouse, and a keyboard for inputting various instructions, commands, information, selections, and settings from an operator (user) to a main body of the medical information processing apparatus, a touch pad (or a track pad) for performing an input operation by touching an operation screen, and a touch panel display (or a touch screen) in which a display screen and the touch pad are integrated. The input interface 12 is connected to the processing circuitry 15, converts the input operation received from the user into an electrical signal, and outputs the electrical signal to the processing circuitry 15. In this case, the input interface 12 may cause the display 13 to display a user interface (graphical user interface (GUI)) for the user to input various instructions by a physical operation component such as a mouse or a keyboard. Note that, in the present specification, the input interface 12 is not limited to one including a physical operation component. For example, an electrical signal processing circuit that receives an electrical signal corresponding to an input operation from an external input device provided separately from the apparatus and outputs the electrical signal to the processing circuitry 15 is also included in the example of the input interface 12. In the following description, “operation of the input interface 12 by the user” is also referred to as “operation by the user”.

The display 13 includes a display body that displays arbitrary data, an internal circuit that supplies a signal for display to the display body, and peripheral circuits such as a connector and a cable that connect the display and an internal circuit. The display 13 is an example of a display unit.

The communication interface 14 is a circuit for connecting the medical information processing apparatus 1 to a network and communicating with other apparatuses. As the communication interface 14, for example, a network interface card (NIC) can be used. In the following description, description that the communication interface 14 is interposed in communication between the medical information processing apparatus 1 and another apparatus is omitted.

The processing circuitry 15 reads the medical information processing program stored in the memory 11 based on an instruction input by the user via the input interface 12, and controls the medical information processing apparatus 1 according to the medical information processing program. For example, the processing circuitry 15 is a processor that realizes each function of the medical information processing apparatus 1 according to the medical information processing program read from the memory 11. Examples of the functions include a first acquisition function 15a, a second acquisition function 15b, a specifying function 15c, a display control function 15d, a notification function 15e, a reservation function 15f, and the like. Note that each function may be implemented by being distributed to a plurality of processors as appropriate. Alternatively, each function or some of the functions may be executed by another apparatus as appropriate. For example, among the functions, the notification function 15e and the reservation function 15f may be executed by another apparatus (not illustrated). That is, the notification function 15e and the reservation function 15f are optional additional items that are not necessarily included in the medical information processing apparatus 1, and may be omitted from the medical information processing apparatus 1.

Next, the first acquisition function 15a, the second acquisition function 15b, the specifying function 15c, the display control function 15d, the notification function 15e, and the reservation function 15f as the respective functions will be described in order. However, division of functions to be described below is for convenience and can be changed as appropriate. This is because even in a case where processing performed by a certain function is performed by another function, there is no change in that the processing circuitry 15 executes the processing. Note that the division of functions can be changed similarly in each of the following embodiments and modifications.

The first acquisition function 15a acquires the examination purpose information regarding the examination purpose including a description regarding a first disease candidate of the patient. Here, the first disease candidate is a disease assumed by the medical doctor at the time of requesting image examination. The examination purpose includes the description of the first disease candidate that the medical doctor wants to confirm in the image examination. In addition, the first acquisition function 15a acquires the examination purpose information based on at least one of the electronic medical record of the patient, the examination order of the image examination, or the image interpretation report acquired from the DWH 8. Specifically, for example, the first acquisition function 15a acquires the examination purpose information based on at least items of the patient information, the examination purpose, and the suspicion of diagnosis among the items of the patient information, main complaint, previous disease, concurrent disease, internal medicine, family history, physical finding, examination purpose, and suspicion of diagnosis regarding the patient. That is, the first acquisition function 15a acquires the examination purpose information based on each item commonly included in at least the three of the electronic medical record, the examination order, and the image interpretation report among the items included in any of the three of them. Note that the first acquisition function 15a may acquire the electronic medical record, the examination order, and the image interpretation report from a location other than the DWH 8. For example, the first acquisition function 15a may acquire each of the electronic medical record and the examination order from the electronic medical record system and the ordering system included in the HIS 2. Further, the first acquisition function 15a may acquire the image interpretation report from the PACS 5. The first acquisition function 15a is an example of a first acquisition unit.

The second acquisition function 15b acquires the image interpretation information including the description regarding the first disease candidate from the image interpretation report regarding the image examination performed according to the examination purpose. For example, the second acquisition function 15b acquires the image interpretation information based on at least an item of finding among items of the finding, evaluation and interpretation, and image interpretation result included in the image interpretation report. The second acquisition function 15b is an example of a second acquisition unit.

The specifying function 15c specifies the incidental finding described in the image interpretation information acquired by the second acquisition function 15b regarding a second disease candidate different from the first disease candidate. In addition, the specifying function 15c further specifies the description regarding the first disease candidate from the image interpretation information. For example, the specifying function 15c extracts the first disease candidate from the examination purpose information, extracts an image interpretation disease candidate including at least the first disease candidate out of the first disease candidate and the second disease candidate from the image interpretation information, and specifies the incidental finding based on the extracted first disease candidate and the image interpretation disease candidate. Specifically, for example, the specifying function 15c extracts the first disease candidate by analyzing the examination purpose information, and extracts the image interpretation disease candidate by analyzing the image interpretation information. The specifying function 15c is an example of a specifying unit.

The display control function 15d causes the display 13 to display a result specified by the specifying function 15c. Here, the display control function 15d may highlight and display the specified result on the display 13. In addition, the display control function 15d may cause the display 13 to display the specified result so as to distinguish the incidental finding and the description regarding the first disease candidate. In addition, the display control function 15d may cause the display 13 to display that the incidental finding is inconsistent with the examination purpose and the description regarding the first disease candidate is consistent with the examination purpose. The display control function 15d may highlight and display the incidental finding and the description regarding the first disease candidate on the display 13. The display control function 15d is an example of a display control unit.

The notification function 15e notifies the clinical department system 6 regarding the result specified by the specifying function 15c of the specified result and patient information regarding the patient. The notification function 15e is an example of a notification unit.

The reservation function 15f transmits the examination reservation information of the patient to the examination department system 7 regarding the result specified by the specifying function 15c. The reservation function 15f is an example of a reservation unit.

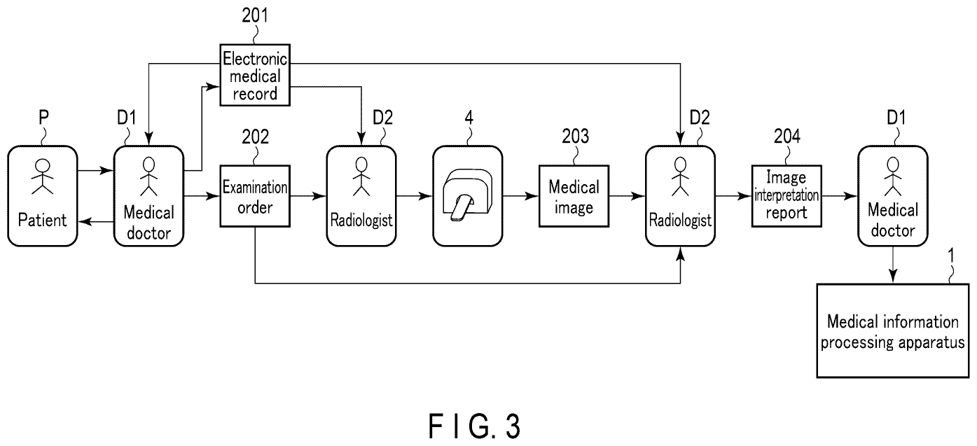

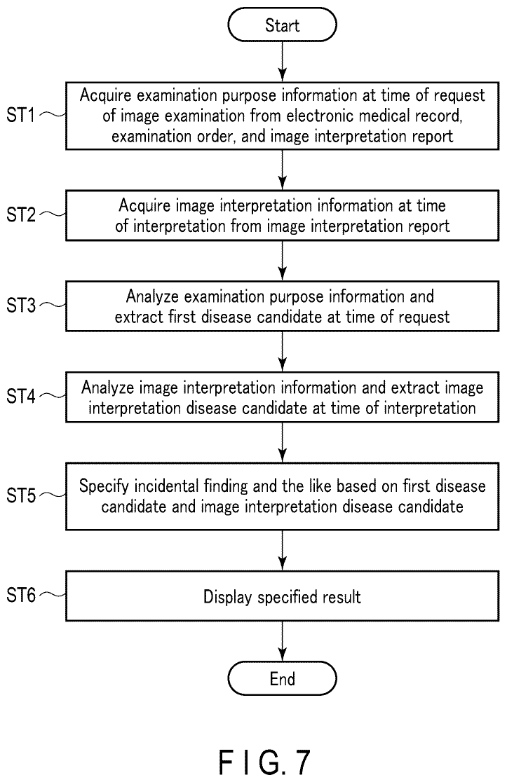

Next, an operation of the medical information processing apparatus configured as described above will be described with reference to FIGS. 3 to 12. First, a flow of image examination will be described with reference to FIGS. 3 to 6. Next, specification of the incidental finding will be described with reference to FIGS. 7 to 12.

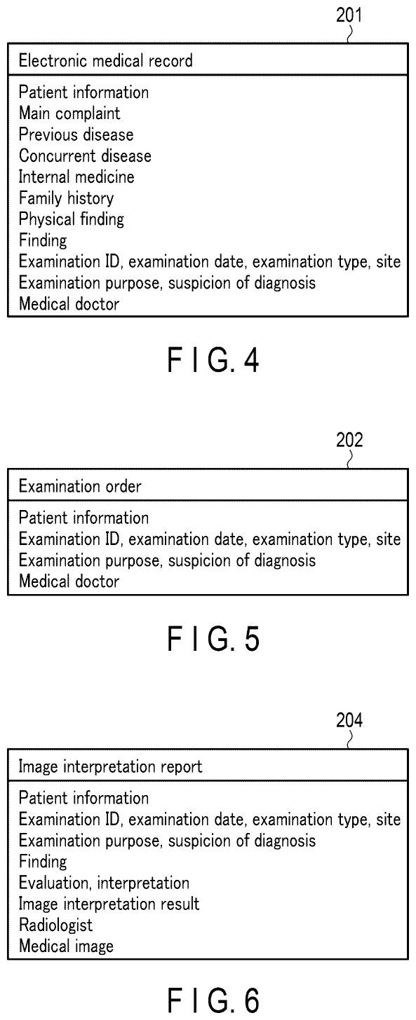

First, the image examination is performed along the flow illustrated from the left side to the right side in FIG. 3. That is, a medical doctor D1 examines a patient P and writes an electronic medical record 201 for each item. Examples of the items of the electronic medical record 201 include, as illustrated in FIG. 4, the patient information, main complaint, previous disease, concurrent disease, internal medicine, family history, physical finding, finding, examination ID, examination date, examination type, site, examination purpose, suspicion of diagnosis, and medical doctor. Note that, in the electronic medical record 201, there is no description regarding the examination purpose such as the examination ID, examination date, examination type, site, examination purpose, and suspicion of diagnosis at a stage before an examination order 202 is issued.

Subsequently, the medical doctor D1 issues the examination order 202 by executing input for each item by the ordering system of the HIS 2, and requests a radiologist D2 (or a technician) to conduct the image examination. Examples of items of the examination order 202 include, as illustrated in FIG. 5, the patient information, examination ID, examination date, examination type, site, examination purpose, suspicion of diagnosis, and medical doctor.

The radiologist D2 conducts the image examination of the patient P using the medical image diagnostic apparatus 4 based on the examination order 202 and the electronic medical record 201 to obtain a medical image 203 of the patient P. In addition, the radiologist D2 creates an image interpretation report 204 by interpreting the medical image 203 while displaying the examination order 202, the electronic medical record 201, and the medical image 203 on the report creation device. Examples of items of the image interpretation report 204 include, as illustrated in FIG. 6, the patient information, examination ID, examination date, examination type, site, examination purpose, suspicion of diagnosis, finding, evaluation, interpretation, image interpretation result, radiologist, and medical image. That is, in a case of creating the image interpretation report 204, the radiologist describes the items of the finding, evaluation, interpretation, and image interpretation result.

Thereafter, the medical doctor displays and confirms the image interpretation report 204 on the medical information processing apparatus 1. At this time, the medical doctor operates the medical information processing apparatus 1 as illustrated in steps ST1 to ST6 of FIG. 7 in order to prevent overlooking of the incidental finding regarding an unexpected disease.

### (Step ST1)

The processing circuitry 15 of the medical information processing apparatus 1 acquires the examination purpose information at the time of requesting the image examination from the electronic medical record 201, the examination order 202, or the image interpretation report 204 stored in the DWH 8.

The examination purpose information includes the description regarding the items of the patient information, examination purpose, and suspicion of diagnosis among the items of the patient information, main complaint, previous disease, concurrent disease, internal medicine, family history, physical finding, examination purpose, and suspicion of diagnosis regarding the patient P. Here, the patient information is items regarding gender, age, and the like. The main complaint is, for example, an item regarding subjective symptoms such as headache. The previous disease is an item regarding a past disease or a medical treatment that has been received. The concurrent disease is an item regarding another disease that occurs at the same time as a certain disease but is not related to the disease. The internal medicine is an item regarding a medicine currently being taken. The family history is, for example, an item regarding a patient family such as a mother having high blood pressure and living together with a wife and a child. A life history is, for example, an item regarding a healthy life such as smoking twenty cigarettes/day×ten years. The physical finding is, for example, an item regarding physical observation such as a blue face with pain. The examination purpose and the suspicion of diagnosis are items regarding the first disease candidate assumed by the medical doctor at the time of request. Note that the patient information, the examination purpose, and the suspicion of diagnosis are described in all the electronic medical record 201, the examination order 202, and the image interpretation report 204.

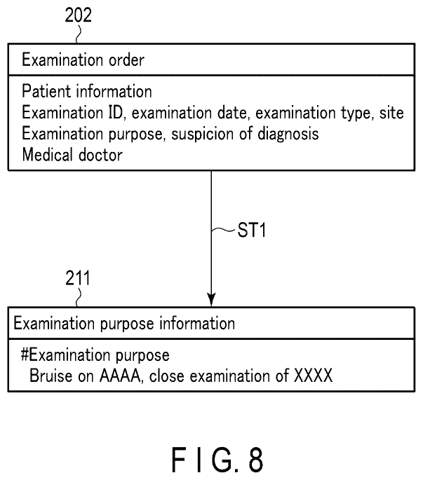

In this example, as illustrated in FIG. 8, the processing circuitry 15 acquires examination purpose information 211 regarding the examination purpose including a description “bruise on AAAA (site name) and close examination of XXXX (first disease candidate)” regarding the first disease candidate of the patient P from the examination order 202. Note that, from the viewpoint of performing diagnosis more accurately, it is desirable that the examination purpose information 211 be acquired from the electronic medical record 201 based on not only the items of the patient information, examination purpose, and suspicion of diagnosis but also many items such as the main complaint, previous disease, concurrent disease, internal medicine, family history, and physical finding. In any case, the processing circuitry 15 stores the acquired examination purpose information 211 in the memory 11.

### (Step ST2)

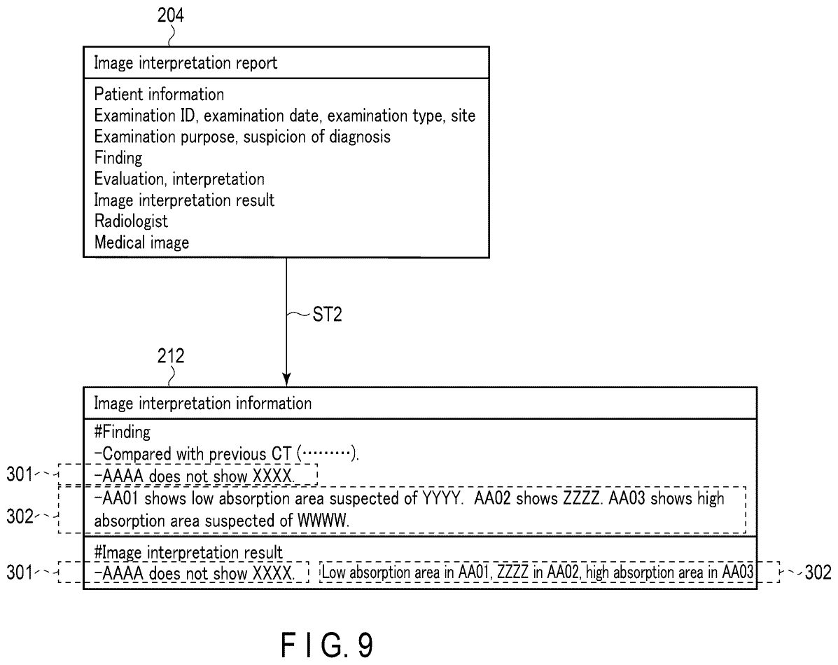

As illustrated in FIG. 9, the processing circuitry 15 acquires image interpretation information 212 including a description regarding the first disease candidate at the time of interpretation from the image interpretation report 204 regarding the image examination performed according to the examination purpose. Specifically, the processing circuitry 15 acquires the image interpretation information 212 based on the items of the finding, evaluation and interpretation, and image interpretation result included in the image interpretation report 204.

Here, in the finding, a state of an inspected site or organ is described in detail. In a case where an abnormality is found, its location, size, shape, density, clarity, and the like are described in the finding. In the evaluation and interpretation, the radiologist describes medical judgment based on the finding of the medical image 203. The evaluation and interpretation may include a meaning of the finding, possible diagnosis, another differential diagnosis, recommended additional examinations, and the like. That is, the evaluation and interpretation are items for analyzing and understanding the result of image interpretation from a medical viewpoint. The image interpretation result is an item regarding a conclusion that summarizes main points of the image interpretation report 204, and may include a proposal of a specific action (for example, additional diagnostic examinations or treatment strategies). The conclusion of the image interpretation result is intended to summarize the image interpretation report 204 and provide key information in an easy-to-read manner for other medical professionals and patients. Note that the evaluation and interpretation and the conclusion of the image interpretation result may include partially overlapping content. In this case, the evaluation and interpretation generally describes a detailed medical analysis and the conclusion is to summarize the information and recommend specific actions. Therefore, the processing circuitry 15 may acquire the image interpretation information 212 based on at least the item of the finding among the items of the finding, evaluation and interpretation, and image interpretation result included in the image interpretation report 204. In the example of FIG. 9, the processing circuitry 15 acquires the image interpretation information 212 based on the items of the finding and image interpretation result. Further, in the example of FIG. 9, the description of items of the image interpretation information 212 includes a description 301 consistent with the first disease candidate and a description 302 regarding the second disease candidate different from the first disease candidate. The description 301 includes, for example, a character string such as “AAAA (site name) does not show XXXX (first disease candidate)”. The description 302 includes, for example, character strings such as “AA01 (part of AAAA) shows a low absorption area suspected of YYYY (second disease candidate). AA02 (another part of AAAA) shows zzzz (another second disease candidate). AA03 (still another part of AAAA) shows a high absorption area suspected of WWWW (still another second disease candidate)” and “a low absorption area in AA01, YYYY in AA02, a high absorption area in AA03”. Note that the description 302 regarding the second disease candidate is a description inconsistent with the first disease candidate. That is, “inconsistent” does not mean to deny the first disease candidate, but means not targeting the first disease candidate. In addition, “consistent” means targeting the first disease candidate, and includes either affirmation or negation of the first disease candidate.

### (Steps ST3 to ST5)

In steps ST3 to ST5, the processing circuitry 15 specifies the incidental finding described in the acquired image interpretation information 212 regarding the second disease candidate different from the first disease candidate. In addition, the processing circuitry 15 further specifies the description regarding the first disease candidate from the image interpretation information 212. Hereinafter, each step of such steps ST3 to ST5 will be individually described.

### (Step ST3)

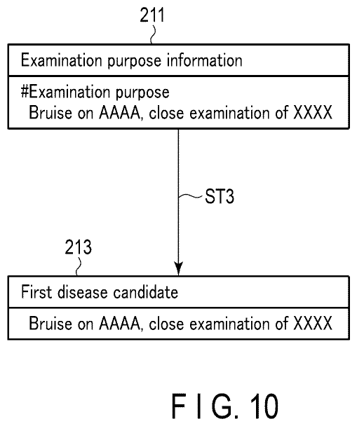

As illustrated in FIG. 10, the processing circuitry 15 extracts a first disease candidate 213 from the examination purpose information 211. For example, the processing circuitry 15 extracts the first disease candidate 213 assumed at the time of request by analyzing the examination purpose information 211 acquired in step ST1. Specifically, for example, the processing circuitry 15 specifies an assumed disease, disease site, and symptom by performing morphological analysis, syntax analysis, semantic analysis, or context analysis for the examination purpose information 211. In addition, the processing circuitry 15 extracts the specified disease, disease site, and symptom as the first disease candidate 213.

### (Step ST4)

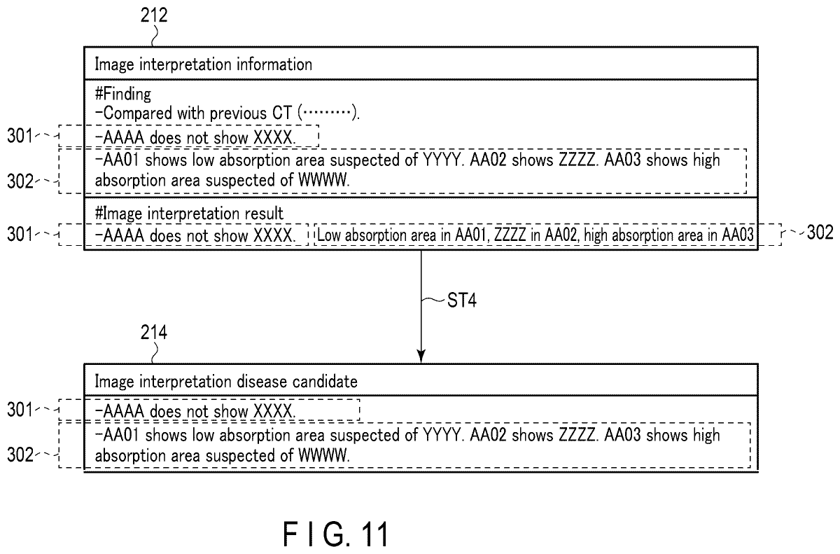

As illustrated in FIG. 11, the processing circuitry 15 extracts an image interpretation disease candidate 214 including at least the first disease candidate out of the first disease candidate and the second disease candidate from the image interpretation information 212. For example, the processing circuitry 15 extracts the image interpretation disease candidate 214 at the time of interpretation by analyzing the image interpretation information 212 acquired in step ST2. Specifically, for example, the processing circuitry 15 specifies the assumed disease, disease site, and symptom by performing morphological analysis, syntax analysis, semantic analysis, or context analysis for the image interpretation information 212. In addition, the processing circuitry 15 extracts the specified disease, disease site, and symptom as the image interpretation disease candidate 214.

### (Step ST5)

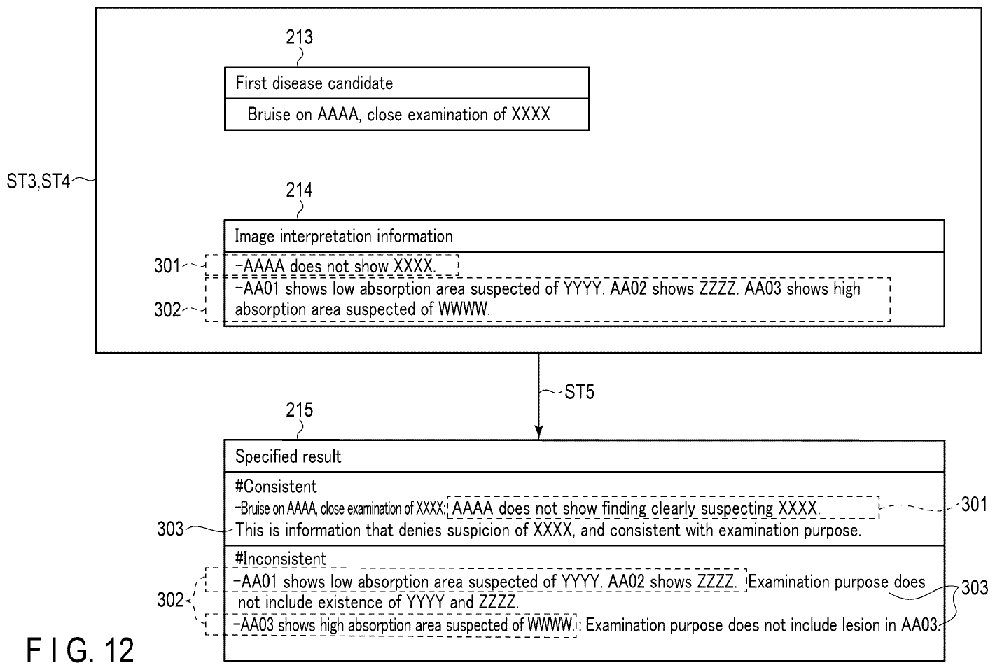

As illustrated in FIG. 12, the processing circuitry 15 specifies the incidental finding and the like based on the first disease candidate 213 acquired in step ST3 and the image interpretation disease candidate 214 acquired in step ST4. Specifically, for example, the processing circuitry 15 specifies the description 301 consistent with the first disease candidate 213 at the time of request in the description included in the image interpretation disease candidate 214 at the time of interpretation. In addition, in the case where the image interpretation disease candidate 214 includes the incidental finding that is the description 302 regarding the second disease candidate different from the first disease candidate, the processing circuitry 15 specifies the incidental finding. Note that the description 302 regarding the second disease candidate is a description inconsistent with the first disease candidate 213. That is, the processing circuitry 15 distinguishes and specifies the description 301 consistent with the first disease candidate 213 and the incidental finding in the description 302 inconsistent with the first disease candidate 213. At this time, the processing circuitry 15 may display, on the display 13, a comment 303 indicating that the incidental finding is inconsistent with the examination purpose and the description regarding the first disease candidate is consistent with the examination purpose.

### (Step ST6)

The processing circuitry 15 may display the result specified in step ST5 on the display 13. For example, the processing circuitry 15 displays, on the display 13, the description 301 consistent with the first disease candidate and the incidental finding that is the description 302 inconsistent with the first disease candidate to be distinguished from each other.

For example, the display 13 displays a first display region including an item “#consistent” and the description 301 consistent with the first disease candidate and a second display area including an item “#inconsistent” and the incidental finding that is the inconsistent description 302 side by side. As a result, the description 301 and the incidental finding in the description 302 are separately displayed.

Further, the processing circuitry 15 may highlight the specified result and display the specified result on the display 13. In this case, the processing circuitry 15 displays, on the display 13, the description 301 and the incidental finding of the description 302 in the same or different colors, for example. For example, in a case where background underlay is white, the processing circuitry 15 may control the display 13 to highlight the description 301 with blue characters, highlight the incidental finding in the description 302 with red characters, and display the remaining description with black characters. In addition, the processing circuitry 15 may highlight and display, on the display 13, a region near the character so as to color the region with a marker, instead of highlighting the character by coloring.

In any case, the description 301 consistent with the first disease candidate and the incidental finding that is the description 302 inconsistent with the first disease candidate are displayed on the display 13. Therefore, in a case of confirming the image interpretation report 204, the medical doctor focuses on the incidental finding regarding an unexpected disease, so that it is possible to prevent overlooking of the incidental finding.

Thereafter, the processing circuitry 15 notifies the clinical department system 6 regarding the specified result of the specified result and the patient information of the patient P in response to the operation of the user such as the medical doctor. The clinical department system 6 accumulates and manages the reported content.

This will be described by a comparative example that does not use the present embodiment. In the comparative example, in a case where the patient P having a disease in the brain and the heart develops cerebral infarction, the medical doctor D1 may overlook the disease in the heart since focusing only on the brain. Further, in the comparative example, the medical doctor D1 may not be able to contact the medical doctor of circulatory system. To cope with the case, the processing circuitry 15 specifies a heart disease candidate as an incidental finding and notifies the clinical department system 6 of the circulatory system of the heart disease candidate. The clinical department system 6 of circulatory system accumulates the reported content and transmits the contents to the medical doctor of circulatory system. As a result, it is possible to reduce overlook or communication omission between different clinical departments.

In addition, the processing circuitry 15 transmits the examination reservation information of the patient P to the examination department system 7 regarding the specified result in response to the operation of the user such as the medical doctor as necessary. The examination department system 7 receives the examination reservation information and transfers the examination reservation information to the examination apparatus. In addition, the examination department system 7 transmits the examination implementation information generated by the examination apparatus to the electronic medical record system included in the HIS 2 as a result of the examination.

As described above, according to the first embodiment, the processing circuitry 15 acquires the examination purpose information 211 regarding the examination purpose including the description regarding the first disease candidate of the patient P. The processing circuitry 15 acquires the image interpretation information 212 including the description regarding the first disease candidate from the image interpretation report 204 regarding the image examination performed according to the examination purpose. The processing circuitry 15 specifies the incidental finding described in the acquired image interpretation information 212 regarding the second disease candidate different from the first disease candidate. The processing circuitry 15 may display the specified result on the display 13. As described above, with the configuration in which the processing circuitry 15 specifies the incidental finding from the image interpretation information 212 acquired from the image interpretation report 204 and displays the specified result, it is possible to specify the incidental finding and give attention to the displayed incidental finding. In addition, according to the first embodiment, unlike conventional technology, a check system by a third party is unnecessary, so that it is possible to suppress an increase in cost.

Further, according to the first embodiment, the processing circuitry 15 may highlight the specified result and display the specified result on the display 13. In this case, it is possible to further give attention to the highlighted incidental finding in addition to the above-described effects.

Furthermore, according to the first embodiment, the processing circuitry 15 further specifies the description regarding the first disease candidate 213. Therefore, it is possible to easily confirm authenticity of the assumption for the first disease candidate 213 that has been assumed by the medical doctor in addition to the above-described effects.

In addition, according to the first embodiment, the processing circuitry 15 displays, on the display 13, the specified result so as to distinguish the incidental finding and the description regarding the first disease candidate. As a result, it is possible to easily confirm the incidental finding and the description regarding the first disease candidate in addition to the above-described effects.

According to the first embodiment, the processing circuitry 15 may display, on the display 13, a comment 303 indicating that the incidental finding is inconsistent with the examination purpose and the description regarding the first disease candidate is consistent with the examination purpose. As a result, it is possible to linguistically confirm a relationship between the incidental finding and the description regarding the first disease candidate with respect to the examination purpose in addition to the above-described effects.

In addition, according to the first embodiment, the processing circuitry 15 highlights and displays, on the display 13, the incidental finding and the description regarding the first disease candidate. As a result, it is possible to further give attention to the highlighted incidental finding and the highlighted description regarding the first disease candidate in addition to the ab-described effects.

According to the first embodiment, the processing circuitry 15 extracts the first disease candidate 213 from the examination purpose information 211. The processing circuitry 15 extracts the image interpretation disease candidate 214 including at least the first disease candidate 213 out of the first disease candidate 213 and the second disease candidate from the image interpretation information 212. The processing circuitry 15 specifies the incidental finding based on the extracted first disease candidate 213 and the image interpretation disease candidate 214. As a result, it is possible to easily and reliably implement the specification step by step like a step-by-step method in addition to the above-described effects.

Further, according to the first embodiment, the processing circuitry 15 extracts the first disease candidate 213 by analyzing the examination purpose information 211, and extracts the image interpretation disease candidate 214 by analyzing the image interpretation information 212. Therefore, in addition to the above-described effects, for example, it can be realized by using natural language processing techniques such as morphological analysis, syntax analysis, semantic analysis, and context analysis.

In addition, according to the first embodiment, the processing circuitry 15 acquires the examination purpose information 211 based on at least one of the electronic medical record 201 of the patient P, the examination order 202 of the image examination, or the image interpretation report 204. Therefore, it is possible to use any information among the electronic medical record 201, the examination order 202, and the image interpretation report 204 to acquire the examination purpose information in addition to the above-described effects.

Further, according to the first embodiment, the processing circuitry 15 acquires the examination purpose information 211 based on at least the items of the patient information, examination purpose, and suspicion of diagnosis among the items of the patient information, main complaint, previous disease, concurrent disease, internal medicine, family history, physical finding, examination purpose, and suspicion of diagnosis regarding the patient P. Therefore, it is possible to obtain the examination purpose information 211 based on minimum necessary information in addition to the above-described effects.

Further, according to the first embodiment, the processing circuitry 15 acquires the image interpretation information 212 based on at least the item of the finding among the items of the finding, evaluation and interpretation, and image interpretation result included in the image interpretation report 204. Therefore, it is possible to obtain the image interpretation information 212 based on minimum necessary information in addition to the above-described effects.

Further, according to the first embodiment, the processing circuitry 15 notifies the clinical department system 6 regarding the specified result of the specified result and the patient information regarding the patient. As a result, for example, another medical doctor for the second disease candidate can proceed with examination of treatment by notifying the clinical department of the second disease candidate of the incidental finding in addition to the above-described effects.

Further, according to the first embodiment, the processing circuitry 15 transmits the examination reservation information of the patient to the examination department system 7 regarding the specified result. Thereby, it is possible to reserve another examination for performing diagnosis for the second disease candidate of the incidental finding in addition to the above-described effects.

### (Modification)

In the first embodiment, the incidental finding of the identified result is displayed on the display 13, but it is not limited to the case. For example, the display control function 15d of the processing circuitry 15 may control the display 13 to further display an importance level for the second disease candidate described in the incidental finding of the specified result based on a relationship between a predetermined disease candidate and the importance level. Further, the importance level may be displayed for the first disease candidate in addition to the second disease candidate. As the relationship between the disease candidate and the importance level, for example, a table associating the disease candidate and the importance level may be stored in the memory 11 in advance, or a description associating the disease candidate and the importance level may be included in a medical information processing program. In addition, the display control function 15d may display the importance level only in a case where a specified result 215 includes a description indicating that a disease candidate is recognized. In other words, in a case where a disease candidate is not recognized in the specified result 215, the importance level of the non-recognized disease candidate may not be displayed. According to such a modification, it is possible to present the importance level of the second disease candidate described in the incidental finding to the medical doctor in addition to the effects of the first embodiment.

### Second Embodiment

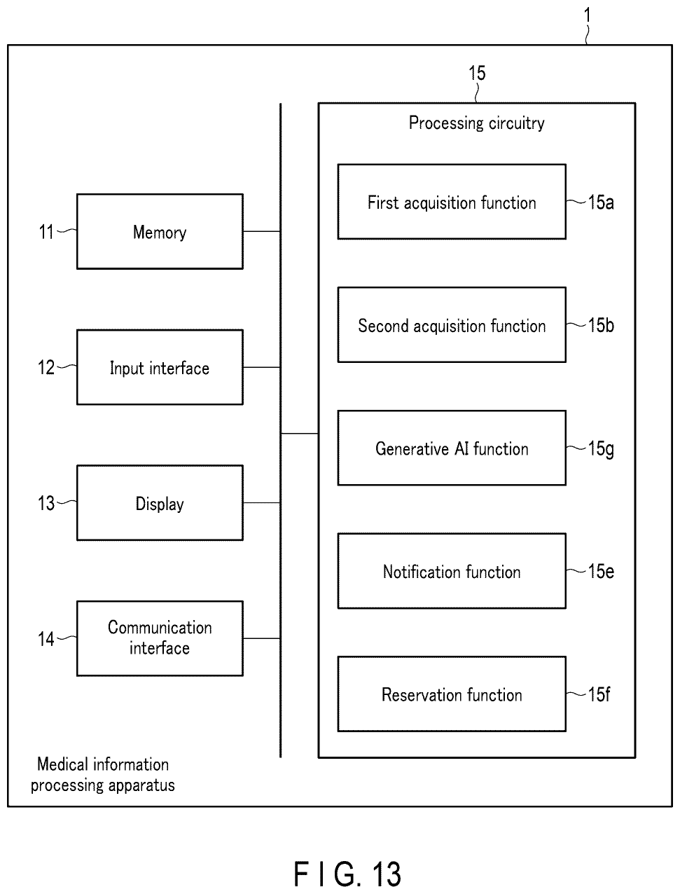

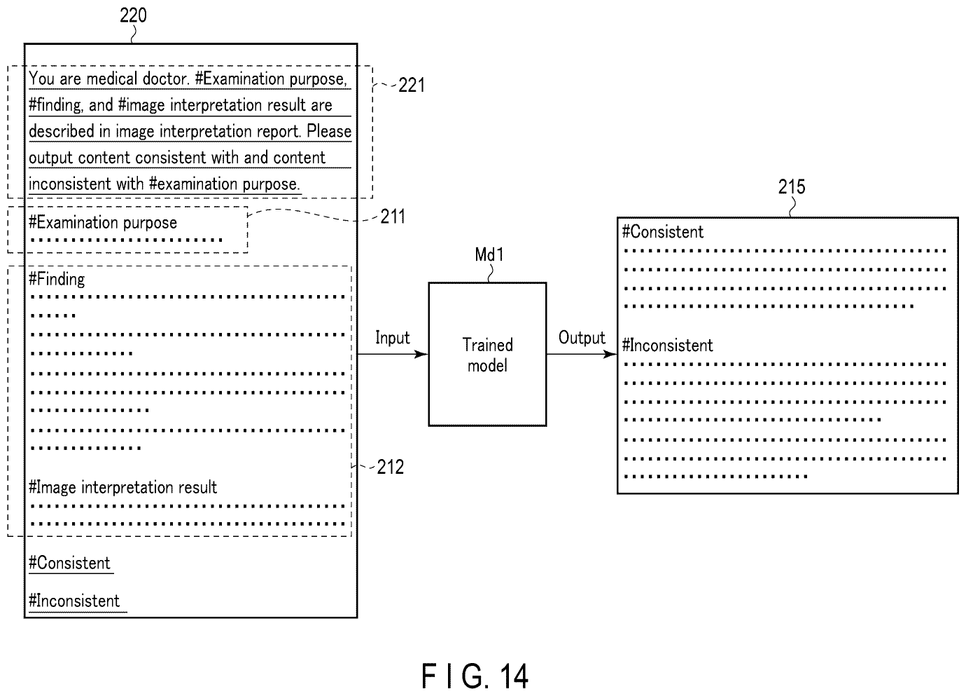

Next, a medical information processing apparatus according to a second embodiment will be described with reference to FIGS. 13 and 14. Note that, in the following description, elements substantially the same as those in the above-described drawings are denoted by the same reference numerals, detailed description thereof is omitted, and different elements will be mainly described. The same similarly applies to the following embodiments.

The second embodiment is a modification of the first embodiment, and is a mode using a generative artificial intelligence (AI) regarding a large language model (LLM) as a configuration for specifying an incidental finding from image interpretation information 212.

Specifically, as illustrated in FIG. 13, processing circuitry 15 includes a generative AI function 15g corresponding to the specifying function 15c and the display control function 15d described above. Note that the generative AI function 15g can be appropriately used as long as it has a configuration including at least a large language model, such as ChatGPT™, for example.

Here, in a case where an instruction for specifying an incidental finding and a description regarding a first disease candidate from examination purpose information 211 and image interpretation information 212, the examination purpose information 211, and the image interpretation information 212 are input, the generative AI function 15g outputs a specified result 215 and displays the result on a display 13. Note that, in examination purpose information 211, the image interpretation information 212, and the specified result 215, character strings similar to those in the first embodiment are described. For example, as illustrated in FIG. 14, the generative AI function 15g inputs an input sentence 220 including an instruction 221, the examination purpose information 211, and the image interpretation information 212 to a trained model Md1, and displays, on the display 13, the specified result 215 output from the trained model Md1. Note that the input sentence 220 may be called a prompt.

The trained model Md1 is, for example, a large language model trained in advance, and generates an output sentence corresponding to the input sentence 220 in a case where the input sentence 220 is input. Note that the output sentence can be changed to a format other than the sentence, such as a table format or a graph format, by specifying an output format by an instruction 221. In addition, in FIG. 14, the instruction 221 is described in a format including an explanatory sentence for describing background information regarding conditions, supplements, and the like and an instruction sentence for obtaining a specified result. Note that the explanatory sentence here is “You are a medical doctor. In image interpretation report, #examination purpose, #finding, #image interpretation result are described”. The instruction sentence is “Please output content consistent with and content inconsistent with #examination purpose”. Note that the present embodiment is not limited thereto, and the instruction 221 can be described in any format. Further, the input sentence 220 is described in a format including the instruction 221, the input data (examination purpose information 211 and image interpretation information 212), and the output format (#consistent and #inconsistent). Further, similarly, the present embodiment is not limited thereto, and the input sentence 220 can be described in any format. Further, in the input sentence 220, an electronic medical record 201, an examination order 202, or an image interpretation report 204 including the content of the examination purpose information 211 may be used instead of the examination purpose information 211. In this case, a first acquisition function 15a that acquires the examination purpose information 211 can be omitted.

Further, in the input sentence 220, the image interpretation report 204 including the content of the image interpretation information 212 may be used instead of the image interpretation information 212. In this case, a second acquisition function 15b that acquires the image interpretation information 212 can be omitted. In addition, the trained model Md1 is stored in a memory 11. Note that the present embodiment is not limited thereto, and for example, the trained model Md1 itself may be preset as the generative AI function 15g in the processing circuitry 15, such as an ASIC or an FPGA. Note that the generative AI function 15g and the trained model Md1 are examples of the generative AI.

Other configurations are similar to the first embodiment.

According to the above configuration, after the examination purpose information 211 and the image interpretation information 212 are acquired by the execution of steps ST1 to ST2, steps ST3 to ST6 are collectively executed, similarly to the above description.

That is, after step ST2, in a case where the input sentence 220 including the instruction 221, the examination purpose information 211, and the image interpretation information 212 is input by an operation of a medical doctor, the processing circuitry 15 outputs and displays the specified result 215 on the display 13. The specified result 215 includes the description consistent with the first disease candidate and the incidental finding that is a description inconsistent with the first disease candidate, as described above. Therefore, in a case of confirming the image interpretation report 204, the medical doctor focuses on the incidental finding regarding an unexpected disease, so that it is possible to prevent overlooking of the incidental finding.

As described above, according to the second embodiment, processing circuitry 15 includes a generative AI function 15g that is a specifying function 15c and a display control function 15d described above. Here, in a case where an instruction for specifying the incidental finding and the description regarding the first disease candidate from the examination purpose information 211 and the image interpretation information 212, the examination purpose information 211, and the image interpretation information 212 are input, the processing circuitry 15 outputs the specified result 215 and displays the result on the display 13. As a result, it is possible to disease the incidental finding and the description regarding the first disease candidate by the generative AI in addition to the above-described effects.

### (Modification)

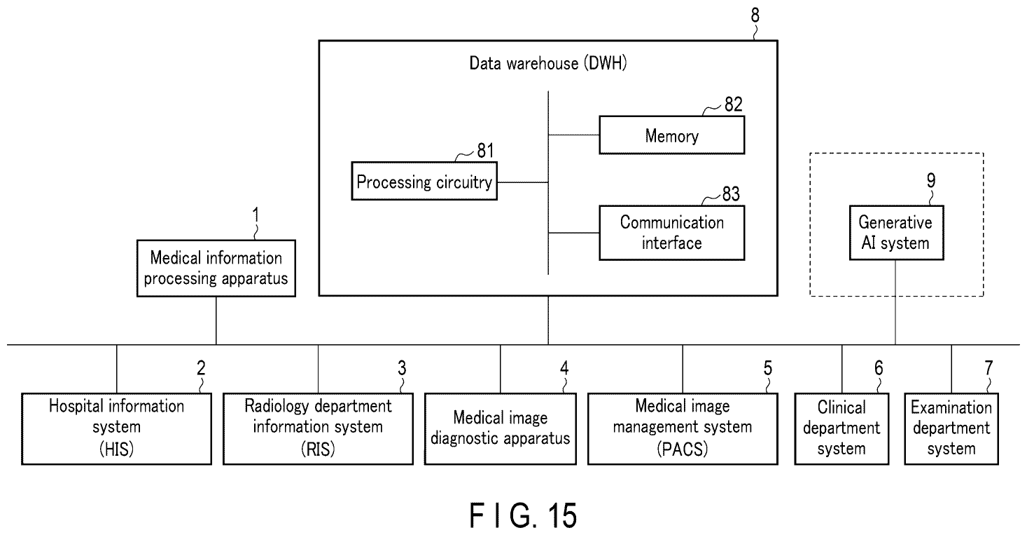

In the second embodiment, a medical information processing apparatus 1 includes the generative AI function 15g, but the present embodiment is not limited thereto. For example, as illustrated in FIG. 15, a generative AI system 9 that implements the generative AI function 15g may be provided outside the medical information processing apparatus 1. The generative AI system 9 may be realized as a server device connected to a network in a hospital, or may be realized as a cloud server device connected to an external network such as the Internet. According to such a modification, it is possible to reduce a load on the medical information processing apparatus 1 to execute the generative AI function 15g in addition to the above-described effects. This modification can be similarly applied to each of the following embodiments.

### Third Embodiment

A third embodiment is a modification of the second embodiment, and is a mode in which an importance level for a specified result 215 is further output and displayed on a display 13.

Here, before outputting the importance level, it is necessary to perform processing of acquiring a time limit for re-examination or re-consultation for each specified result 215 (for each of consistent and inconsistent items) from #finding and #image interpretation result. Note that a short time limit indicates a high importance level. In addition, there are the following three cases (i) to (iii) regarding a description of the time limit in the #finding and the #image interpretation result in image interpretation information 212.
(i) Case where the time limit is described quantitatively (immediately, 7 days, 14 days, 60 days, 180 days, and indefinite).
(ii) Case where the time limit is described qualitatively (short term, medium term, and long term).
(iii) Case where the time limit is not described.

Therefore, in the case of the above (i), for example, processing circuitry 15 uses the quantitative number of days as it is on a horizontal axis (importance level) of a graph and uses consistency and inconsistency on a vertical axis of the graph to graphically display a specified result 215.

Further, in the case of the above (ii), the processing circuitry 15 uses the qualitative short time, medium term, and long term on the horizontal axis (importance level) of the graph and uses the consistency and inconsistency on the vertical axis of the graph to graphically display the specified result 215.

Alternatively, the processing circuitry 15 may define a conversion table in which a qualitative deadline and quantitative days are associated with each other, such as the short term=14 to 28 days, the medium term=60 to 180 days, and the long term=180 to 360 days, and display a graph using the number of days converted from the qualitative time limit on the horizontal axis of the graph.

Further, in the case of the above (iii), the processing circuitry uses the consistency and inconsistency on the vertical axis of the graph without the horizontal axis (importance level) of the graph to graphically display the specified result 215.

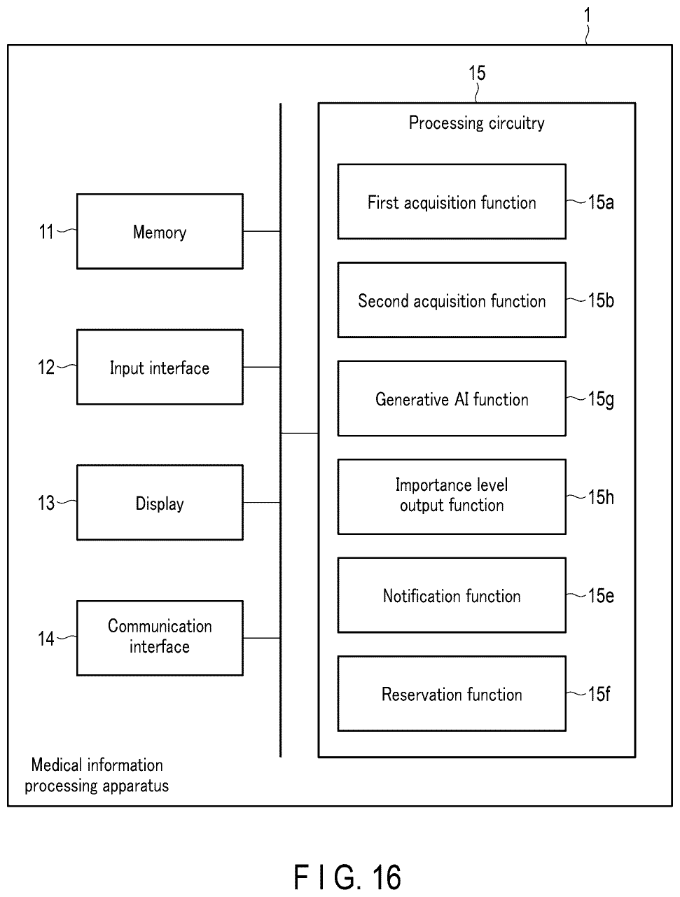

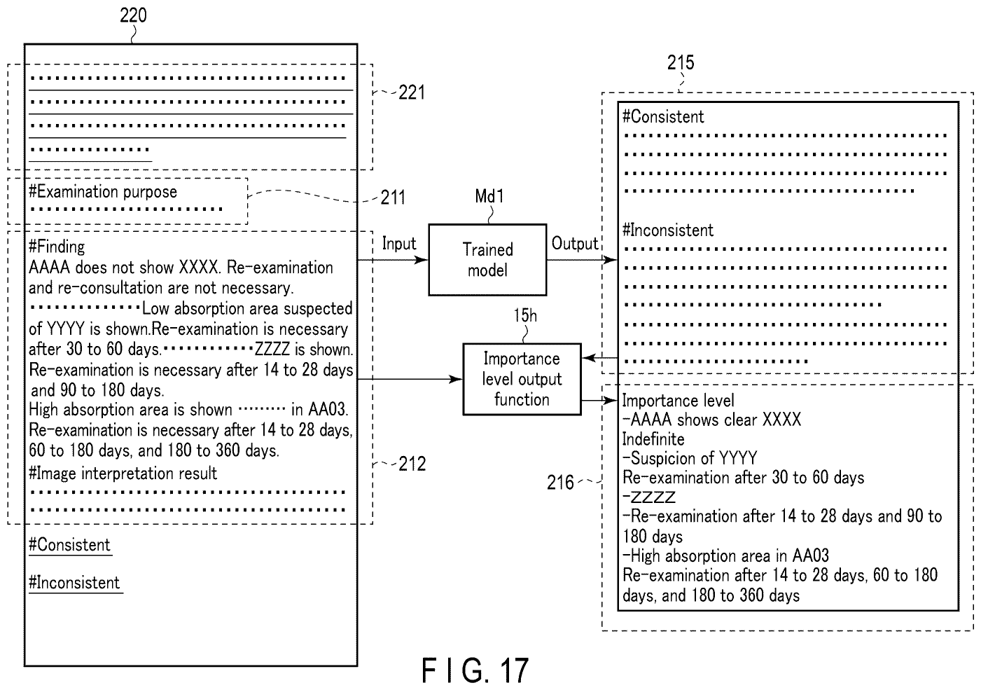

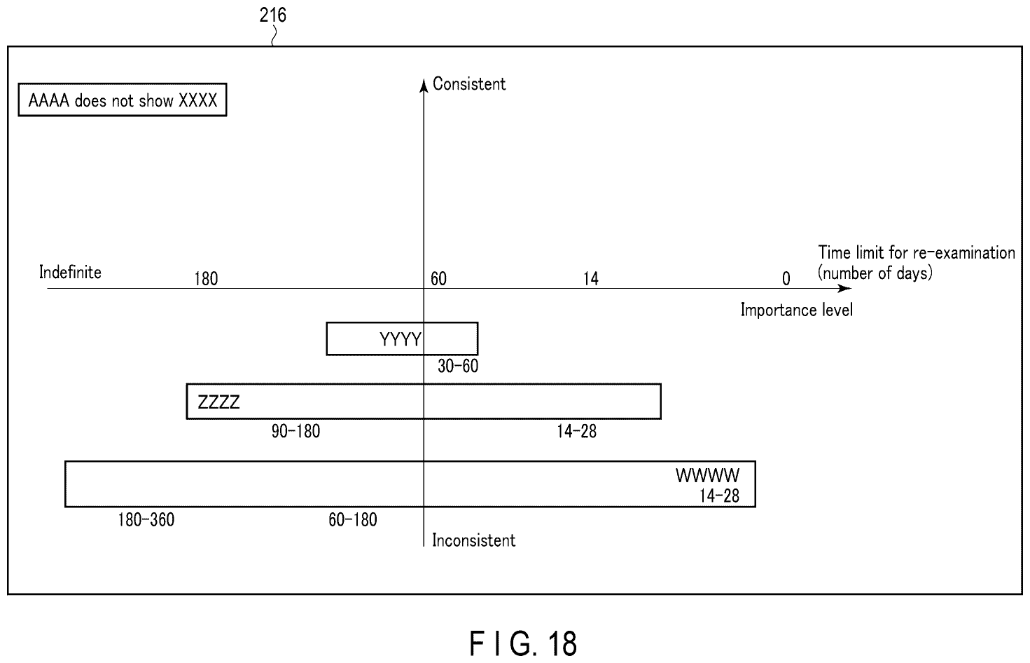

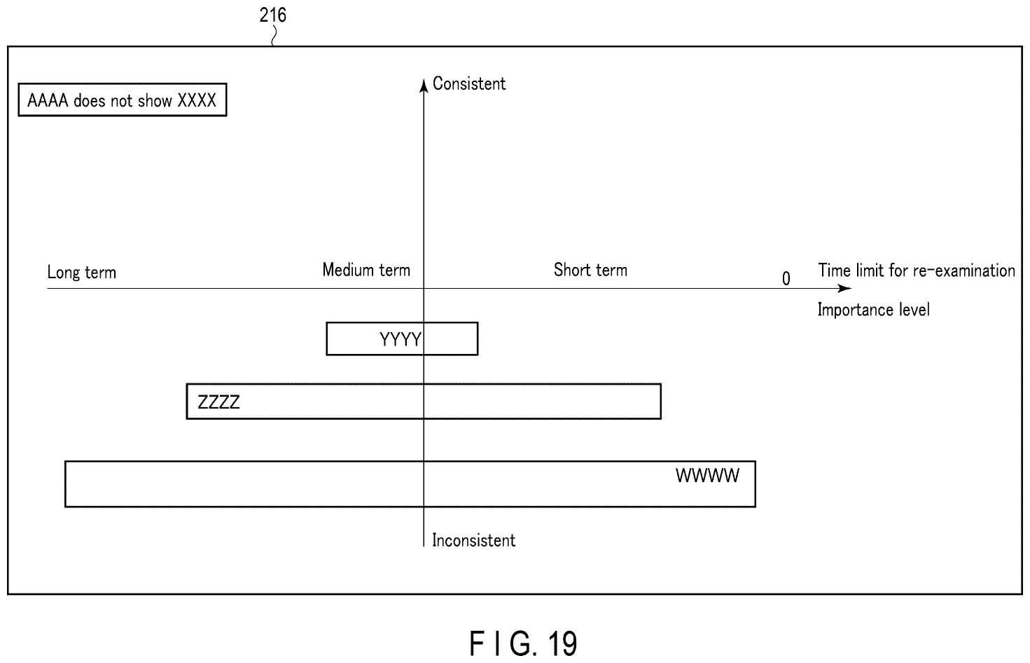

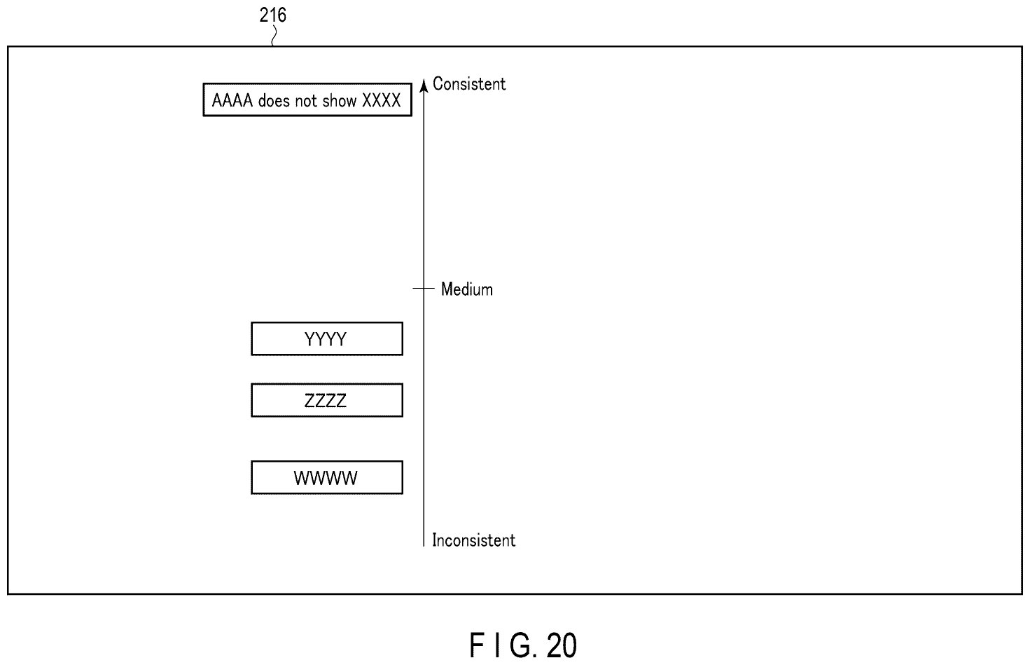

Specifically, as illustrated in FIGS. 16 and 17, the processing circuitry 15 further includes an importance level output function 15h that further outputs an importance level 216 with respect to the specified result 215 based on the image interpretation information 212 and displays the importance level on the display 13. For example, the importance level output function 15h controls the display 13 to further display an importance level 216 for a second disease candidate described in an incidental finding of the specified result 215 based on a relationship between a predetermined disease candidate and the importance level. Note that, in FIG. 17, the importance level is displayed on the display 13 in the form of a sentence, but the present embodiment is not limited thereto. For example, the processing circuitry 15 may change the output format of the importance level 216 to a graph format as illustrated in any of FIGS. 18 to 20. For example, as illustrated in FIGS. 18 and 19, in the graph, the vertical axis may represent the consistency and inconsistency, and the horizontal axis may represent the importance level. Note that FIG. 18 corresponds to the above-described case (i), and FIG. 19 corresponds to the above-described case (ii). Further, FIG. 20 corresponds to the above-described case (iii). The importance level output function 15h is an example of an importance level output unit.

Accordingly, as compared with the above description, the image interpretation information 212 includes a description regarding the importance level indicating that the re-examination is necessary after a predetermined number of days for each disease candidate. Note that the present embodiment is not limited thereto, and the image interpretation information 212 may include a description regarding the importance level indicating that the re-examination and re-consultation are unnecessary for each disease candidate. Furthermore, the description regarding the importance level included in the image interpretation information 212 is described in an image interpretation report 204 in advance by a radiologist and acquired as a part of the image interpretation information 212. In FIG. 17, the following four descriptions are regarding the importance level in the image interpretation information 212. The first description is a description of “the re-examination and re-consultation are unnecessary” for XXXX (first disease candidate). The second description is a description of “the re-examination is necessary after 30 to 60 days” for YYYY (second disease candidate). The third description is a description of “the re-examination after 14 to 28 days and 90 to 180 days is necessary” for Zzzz (second disease candidate). The fourth description is “re-examination is required after 14 to 28 days, 60 to 180 days, and 180 to 360 days” for the high absorption area found in AA03 (the site name of the incidental finding).

Other configurations are similar to the second embodiment.

According to the above configuration, after examination purpose information 211 and the image interpretation information 212 are acquired by execution of steps ST1 to ST2, steps ST3 to ST6 are collectively executed, similarly to the above description.

That is, after step ST2, in a case of the input sentence 220 including the instruction 221, the examination purpose information 211, and the image interpretation information 212 is input by an operation of a medical doctor, the processing circuitry 15 outputs and displays the specified result 215 and the importance level 216 on the display 13. The specified result 215 includes the description consistent with the first disease candidate and the incidental finding that is a description inconsistent with the first disease candidate, as described above. The importance level 216 includes, for example, a description of a predetermined number of days “indefinite” for a description consistent with the first disease candidate and descriptions of predetermined numbers of days “30 to 60 days”, “14 to 28 days and 90 to 180 days”, and “14 to 28 days, 60 to 180 days, and 180 to 360 days” for the incidental findings that are descriptions inconsistent with the first disease candidate. Therefore, in a case of confirming the image interpretation report 204, the medical doctor focuses on the incidental findings regarding an unexpected disease and the importance level, so that it is possible to further prevent overlooking of the incidental findings.

According to the third embodiment as described above, the processing circuitry 15 further outputs the importance level 216 with respect to the specified result 215 based on the image interpretation information 212 and displays the importance level on the display 13, using the importance level output function 15h. Therefore, it is possible to present the importance level of the second disease candidate described in the incidental finding to the medical doctor in addition to the above-described effects.

### (Modification)

In the third embodiment, in the graph format of the importance level, the vertical axis represents the consistency and inconsistency, and the horizontal axis represents the importance level, but the present embodiment is not limited thereto. For example, in FIGS. 18 to 20, the vertical axis of the graph may be turned upside down. Similarly, the horizontal axis of the graph may be horizontally reversed. In addition, the vertical axis and the horizontal axis of the graph may be exchanged. Alternatively, the importance level may be displayed only in the case of inconsistency. In any case, according to such a modification, similarly to the effects of the third embodiment, it is possible to present the importance level of the disease candidate in a graph form in a visually easy-to-understand manner.

### Fourth Embodiment

A fourth embodiment is a modification of the second embodiment, and is a mode for preventing overlooking of important findings regarding examination. Specifically, for example, it is assumed that there is a workflow in which a gastroenterologist creates an endoscopic report (image interpretation report) for an endoscopic examination order from a medical doctor, and the medical doctor confirms the endoscopic report. Here, a case is assumed in which, before the creation of the endoscopic report, the gastroenterologist issues a pathological examination order of a polyp found by endoscopic examination to a pathology examiner, and the pathology examiner creates a pathological report including an important finding that a pathological examination result of the polyp indicates malignancy. In this case, if the gastroenterologist creates the endoscopic report in a state of overlooking the pathological report, the content of the pathological examination order and the important finding that the pathological examination result of the polyp is malignant are not described in the endoscopic report. Therefore, the medical doctor cannot grasp the important finding that the pathological examination result of the polyp is malignant, and does not treat the polyp. Note that the medical doctor does not issue the pathological examination order, and thus does not notice the presence of the pathological examination result. Also note that the case where the medical doctor, who does not issue the pathological examination order, does not notice the presence of the pathological examination result and thus overlooks the important finding of the pathological examination result is more common than the case where the gastroenterologist overlooks the pathological examination result. Meanwhile, if the malignant polyp is untreated, there is a possibility that the polyp grows into cancer, and thus it is necessary to notify the medical doctor that there is an overlook of the important finding in some way.

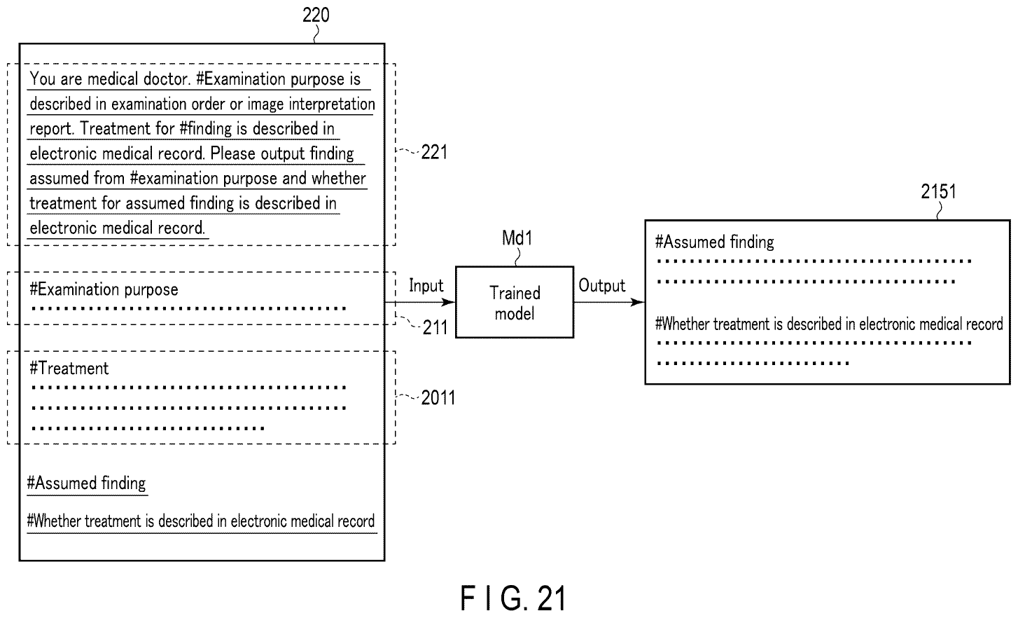

Accordingly, in a case where an instruction for outputting a finding assumed from examination purpose information 211 or image interpretation information 212 and whether or not a treatment for the assumed finding is described in an electronic medical record 201, the examination purpose information 211 or the image interpretation information 212 corresponding to the instruction, and the electronic medical record 201 are input, a generative AI function 15g of processing circuitry 15 illustrated in FIG. 13 outputs the assumed finding and whether or not the treatment is described in the electronic medical record 201, and displays the output on a display 13, in addition to the above-described functions. For example, as illustrated in FIG. 21, the generative AI function 15g inputs an input sentence 220 including an instruction 221, the examination purpose information 211, and a description 2011 of the treatment of the electronic medical record 201 to a trained model Md1, and displays, on the display 13, an output sentence 2151 output from the trained model Md1. The trained model Md1 is, for example, a large language model (LLM) trained in advance, and generates the output sentence 2151 corresponding to the input sentence 220 in a case where the input sentence 220 is input, similarly to the above description. In FIG. 21, the instruction 221 is described in a format including an explanatory sentence and an instruction sentence as described above. Note that the explanatory sentence here is “You are a medical doctor. The examination order or the image interpretation report describes #examination purpose. The electronic medical record describes a treatment for #finding”. The instruction sentence is “Please output the finding assumed from #examination purpose, and whether or not the treatment for the assumed finding is described in the electronic medical record”. Note that the description content of “#examination purpose” can be acquired from both the examination purpose information 211 and the image interpretation information 212. Further, the input sentence 220 is described in a format including the instruction 221, input data (the examination purpose information 211 and the description 2011 of the treatment in the electronic medical record 201), and an output format (#assumed finding and #whether or not the treatment is described in the electronic medical record). Note that, in the output format, “#whether or not the treatment is described in the electronic medical record” may be changed to “#consistent” or “#inconsistent” described above, because presence of the description indicates consistency between the treatment and the electronic medical record, and absence of the description indicates inconsistency. Alternatively, “#whether or not the treatment is described in the electronic medical record” may be changed to any output format corresponding to the consistency or inconsistency between the treatment and the electronic medical record, such as “#assumed finding consistent with the electronic medical record” or “#assumed finding not described in the electronic medical record”. In the output sentence 2151, content based on the instruction 221 is described in the output format.

Other configurations are similar to the second embodiment.

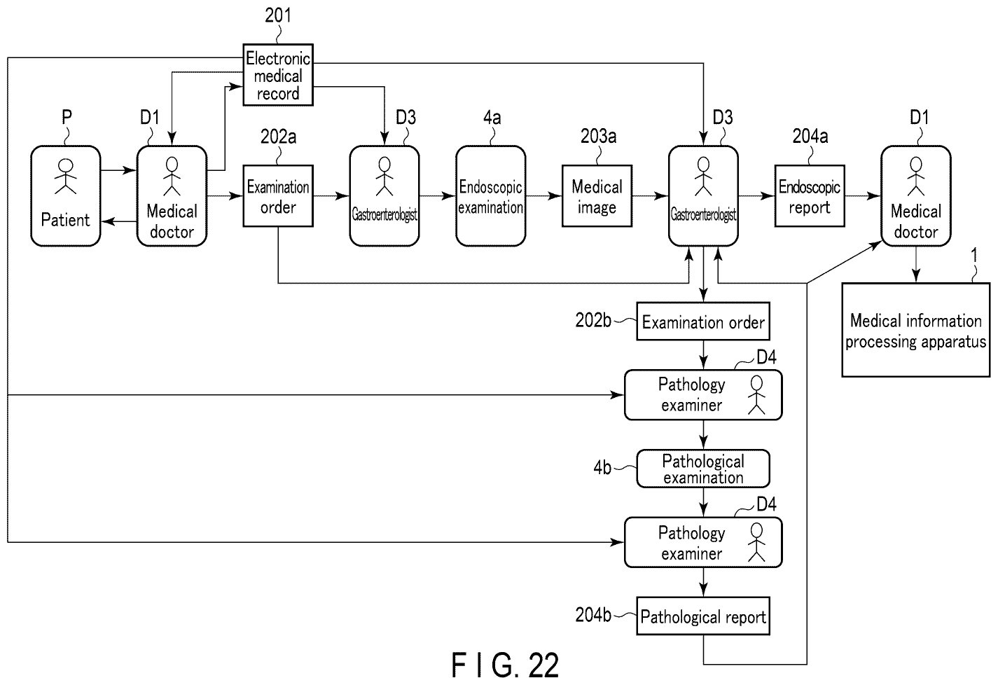

Next, an operation of a medical information processing apparatus 1 configured as described above will be described with reference to the schematic diagrams of FIGS. 22 to 26. In the following description, to prevent overlooking of an important finding regarding an examination, a case where the examination is an endoscopic examination, and the important finding is a finding including that a pathological examination result of a polyp found by the endoscopic examination indicates malignancy will be described as an example.

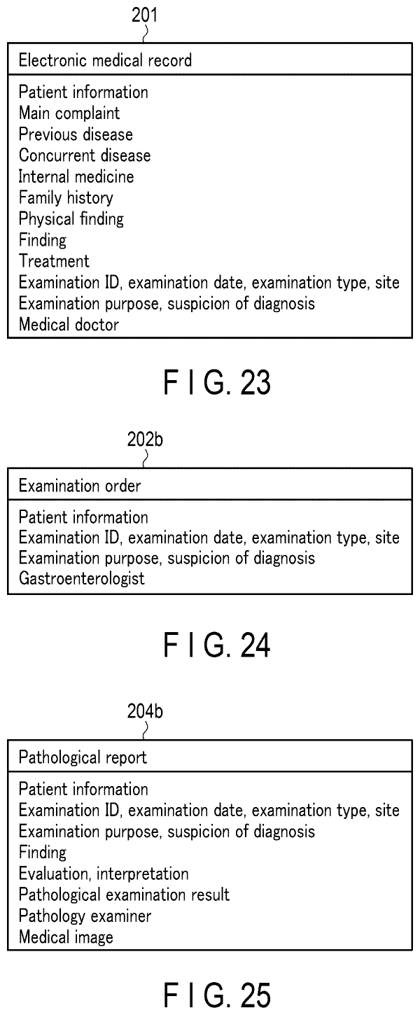

First, the endoscopic examination is performed along the flow illustrated from the left side to the right side in FIG. 22. That is, a medical doctor D1 examines a patient P and writes an electronic medical record 201 for each item. Examples of the electronic medical record 201 include, as illustrated in FIG. 23, patient information, main complaint, previous disease, concurrent disease, internal medicine, family history, physical finding, finding, treatment, examination ID, examination date, examination type, site, examination purpose, suspicion of diagnosis, and medical doctor.

Subsequently, the medical doctor D1 issues an examination order 202a by executing input for each item by an ordering system of an HIS 2, and requests a gastroenterologist D3 to conduct an endoscopic examination that is one of image examinations. Items of the examination order 202a are similar to those in FIG. 5, for example, but the content of the items relate to the endoscopic examination. Note that the examination order 202a of the endoscopic examination does not include a description regarding a polyp because the order was issued before the polyp was found by the endoscopic examination.

The gastroenterologist D3 conducts an endoscopic examination 4a of the patient P using an endoscope (not illustrated) based on the examination order 202a and the electronic medical record 201, and obtains a medical image 203a of the patient. In addition, since the gastroenterologist D3 has found the polyp during the endoscopic examination, the gastroenterologist issues an examination order 202b by executing an input to each item, and requests a pathology examiner D4 to perform a pathological examination for the polyp, using the ordering system of the HIS 2. Examples of items of the examination order 202b include, as illustrated in FIG. 24, patient information, examination ID, examination date, examination type, site, examination purpose, suspicion of diagnosis, and gastroenterologist.

The pathology examiner D4 conducts a pathological examination 4b for the polyp of the patient P using a pathological examination device (not illustrated) based on the examination order 202b and the electronic medical record 201. Further, the pathology examiner D4 creates a pathological report 204b while displaying the examination order 202b, the electronic medical record 201, and the medical image of the polyp on a report creation device. Examples of items of the pathological report 204b include, as illustrated in FIG. 25, the patient information, examination ID, examination date, examination type, site, examination purpose, suspicion of diagnosis, finding, evaluation, interpretation, pathological examination result, pathology examiner, and medical image. That is, in a case of creating the pathological report 204b, the pathology examiner describes the items of the finding, evaluation, interpretation, and pathological examination result. Here, it is assumed that the finding of the pathological report 204b includes that the pathological examination result of the polyp indicates malignancy.

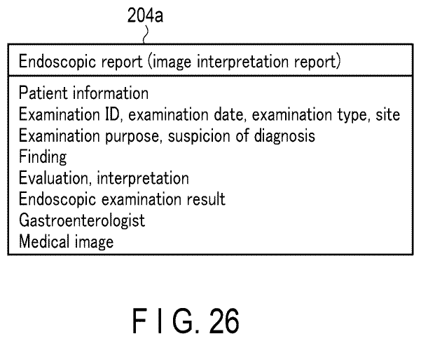

In addition, the gastroenterologist D3 creates an endoscopic report 204a as an image interpretation report by interpreting the medical image 203a while displaying the examination order 202a, the electronic medical record 201, and the medical image 203a on the report creation device. Examples of items of the endoscopic report 204a include, as illustrated in FIG. 26, the patient information, examination ID, examination date, examination type, site, examination purpose, suspicion of diagnosis, finding, evaluation, interpretation, endoscopic examination result, gastroenterologist, and medical image. To supplement, the gastroenterologist D3 creates the endoscopic report 204a based on the result of the endoscopic examination 4a in a state of overlooking the examination order 202b and the pathological report 204b regarding the pathological examination. Therefore, the endoscopic report 204a does not include the finding that the pathological examination result of the polyp indicates malignancy.

The medical doctor D1 displays the endoscopic report 204a on the medical information processing apparatus 1 and confirms the report. At this time, since the medical doctor D1 confirms the endoscopic report 204a not including the indication that the polyp is malignant, the medical cannot grasp that the polyp is malignant and does not perform the treatment for the polyp. Therefore, the electronic medical record 201 includes the item “treatment”, but does not describe the treatment for the finding of the polyp.

At a later date, the medical doctor D1 operates the medical information processing apparatus 1 at the time of consultation of the patient.

For example, the processing circuitry 15 of the medical information processing apparatus 1 outputs the output sentence 2151 based on the instruction 221 and displays the output sentence on the display 13 in a case where the input sentence 220 including the instruction 221, the examination purpose information 211, and the description 2011 of the treatment of the electronic medical record 201 is input by the operation of the medical doctor D1, as illustrated in FIG. 21. That is, the output sentence 2151 includes description content for “#assumed finding” and description content for “#whether or not the treatment is described in the electronic medical record”. Specifically, for example, the description content for “#assumed finding” includes that the finding that the pathological examination result of the polyp is malignant is assumed. The description content for “#whether or not the treatment is described in the electronic medical record” includes that the treatment for the assumed finding is not described.

By the display of the output sentence 2151, the medical doctor understands that there is no treatment for the finding assumed from the examination purpose of the endoscopic examination in a case of confirming the endoscopic report 204a based on the output sentence 2151. Thereafter, the medical doctor can prevent overlooking of the important finding regarding the examination by confirming the important finding in the pathological report 204b corresponding to the assumed finding.

As described above, according to the fourth embodiment, the processing circuitry 15 includes the generative AI function 15g. In a case where the instruction 221 for outputting the finding assumed from the examination purpose information 211 or the image interpretation information 212 and whether or not the treatment for the assumed finding is described in the electronic medical record 201, the examination purpose information 211 or the image interpretation information 212 corresponding to the instruction 221, and the electronic medical record 201 are input, the processing circuitry 15 outputs the assumed finding and whether or not the treatment is described in the electronic medical record 201, and displays the output on the display 13. Therefore, it is possible to prevent overlooking of the important finding regarding the examination by confirming the important finding for the assumed finding based on the content output according to the instruction 221.

### (Modification)

In the fourth embodiment, the finding assumed from the #examination purpose described in both the examination order and the image interpretation report is output, but the embodiment is not limited thereto. For example, the finding assumed from the #examination purpose described in the examination order may be output, or the #finding described in the image interpretation report or the finding assumed from the #image interpretation result may be output. That is, a change can be appropriately made as long as the finding assumed from the examination order or the image interpretation report is output. Even in such a modification, effects similar to those of the fourth embodiment can be obtained. Further, this modification can be similarly implemented in each of the following embodiments.

In addition, in the fourth embodiment, by displaying the output sentence 2151, the medical doctor understands that the treatment for the assumed finding is not described in the electronic medical record, but the present embodiment is not limited thereto. For example, in a case where there is no treatment for the assumed finding, a warning may be output in parallel with the output of the output sentence 2151. The warning may include a message indicating that there is no treatment for the assumed finding, or may be display data that highlights a description indicating that there is no treatment for the assumed finding in the output sentence 2151. According to such a modification, it is possible to prevent overlooking of the description that there is no treatment for the assumed finding from the output sentence 2151 in addition to the effects of the fourth embodiment.

In addition, in the fourth embodiment, after the electronic medical record 201 is stored, the input sentence 220 including an instruction to the generative AI function 15g is input and the output sentence 2151 to the medical doctor is output at the time of consultation of the patient, but the present embodiment is not limited thereto. For example, the instruction to the generative AI function 15g and the outputting to the medical doctor may be configured in such a manner that a text up to the characters that have been input is output during when the treatment is being input to the electronic medical record, or the outputting is performed at the time when the input data has been stored in the electronic medical record.

### Fifth Embodiment

A fifth embodiment is a modification of the second embodiment, and is a mode of outputting candidates of A and P from SO among SOAP described in an electronic medical record. Here, “S” is an acronym of subjective data, and corresponds to, for example, a “main complaint” indicating a statement of a patient in electronic medical record 201 illustrated in FIG. 23. “O” is an acronym of objective data, and corresponds to, for example, a “physical finding” indicating an observation result by a doctor in the electronic medical record 201. “A” is an acronym of assessment, and corresponds to, for example, a “finding” indicating a result obtained by interpretation or analysis of S and O in the electronic medical record 201. “P” is an acronym of “plan”, and corresponds to, for example, “treatment” indicating a treatment plan for S, O, and A in the electronic medical record 201. That is, the fifth embodiment is a mode of outputting findings and treatment candidates from a main complaint and physical findings, for example.

Specifically, for example, in a consultation room, a medical doctor inputs a main complaint and physical findings (SO) from a conversation with the patient to the electronic medical record 201. Alternatively, the medical doctor may perform key input of content of the conversation with the patient in an electronic file other than the electronic medical record. In any case, in the consultation room, the medical doctor needs to explain the findings and treatment (AP) based on the conversation with the patient and the like to the patient.

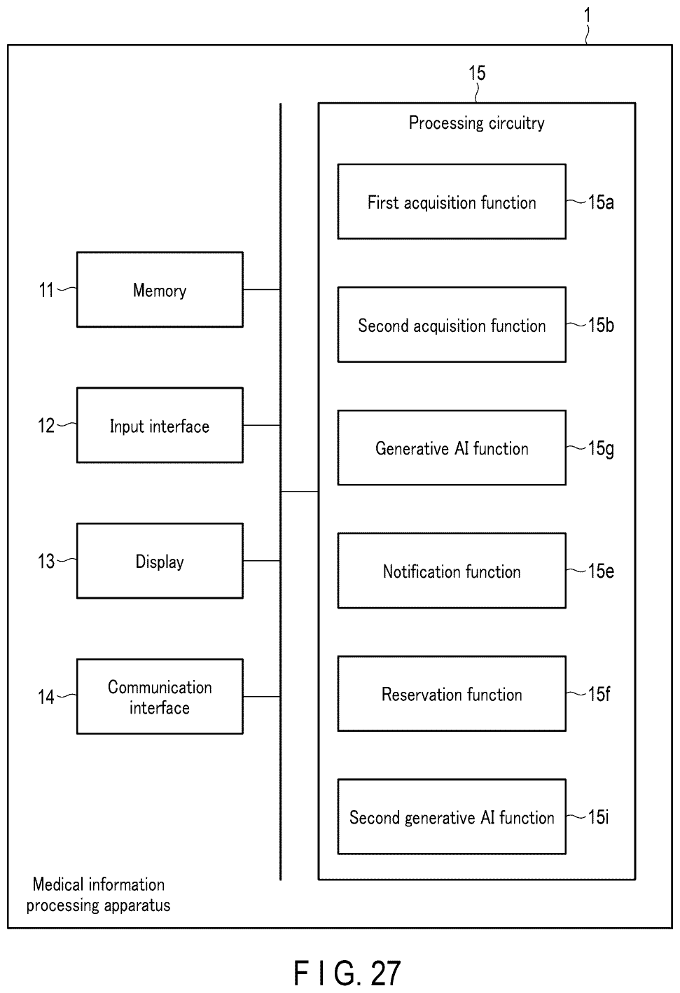

Accordingly, as illustrated in FIG. 27, processing circuitry 15 further includes a second generative AI function 15i different from the above-described generative AI function 15g. Note that the second generative AI function 15i may be referred to as any another name. Alternatively, the above-described generative AI function 15g may be referred to as first generative AI function 15g. In any case, the second generative AI function 15i is an example of a generative AI.

In a case where an instruction for outputting a finding assumed from examination purpose information 211 or image interpretation information 212 and a treatment candidate specified from a main complaint and a physical finding of a patient with respect to the assumed finding, the examination purpose information 211 or the image interpretation information 212 corresponding to the instruction, and the main complaint and the physical finding are input, the second generative AI function 15i outputs and displays the assumed finding and the treatment candidate on a display 13. For example, as illustrated in FIG. 22, the second generative AI function 15i inputs an input sentence 220 including an instruction 222, the examination purpose information 211, and a description 2012 of the main complaint and the physical finding of the electronic medical record 201 to a trained model Md2, and displays, on the display 13, an output sentence 2152 output from the trained model Md2. The trained model Md2 is, for example, a large language model (LLM) trained in advance, and generates the output sentence 2152 corresponding to the input sentence 220 in a case where the input sentence 220 is input, similarly to the above description. In FIG. 22, the instruction 222 is described in a format including an explanatory sentence and an instruction sentence as described above. Note that the explanatory sentence here is “You are a medical doctor. The examination order or the image interpretation report describes #examination purpose. The electronic medical record describes #main complaint and #physical finding”. The instruction sentence is “Please output the finding assumed from the #examination purpose and a treatment candidates specified from the #main complaint and the #physical finding for the assumed finding”. Note that the description content of “#examination purpose” can be acquired from both the examination purpose information 211 and the image interpretation information 212. Furthermore, the description of “#main complaint and #physical finding” is not limited to the acquisition from the electronic medical record 201, and may be acquired from content of the conversation with the patient. Further, the input sentence 220 is described in the format including the instruction 222, input data (the examination purpose information 211 and the description 2012 of the main complaint and the physical finding), and an output format (#assumed finding and #treatment candidate). Note that the output format is not limited to the “#assumed finding” and the “#treatment candidate”. As the output format, for example, any element such as a patient's wish or a standard treatment may be interposed between the assumed finding and the treatment candidate, such as “#the finding assumed from the examination order or the result of the image interpretation report”, “#the patient's wish from the main complaint of the electronic medical record and the physical findings (SO)”, “#the treatment advanced by the doctor from the conversation in the consultation room”, “#the patient's wish from the conversation in the consultation room”, “#the standard treatment for the assumed finding”, and “#the standard treatment and the next treatment candidate from the patient's wish”. In the output sentence 2152, content based on the instruction 222 is described in the output format.

Other configurations are similar to the second embodiment.

According to the above configuration, the medical doctor examines the patient and writes the electronic medical record 201 for each item. At this stage, content including the main complaint and the physical findings (SO) such as patient information, main complaint, previous disease, concurrent disease, internal medicine, family history, physical findings, and medical doctor are written in the electronic medical record 201, but content including the finding and the treatment (AP) is not written. At the time of such consultation, the medical doctor operates a medical information processing apparatus 1.

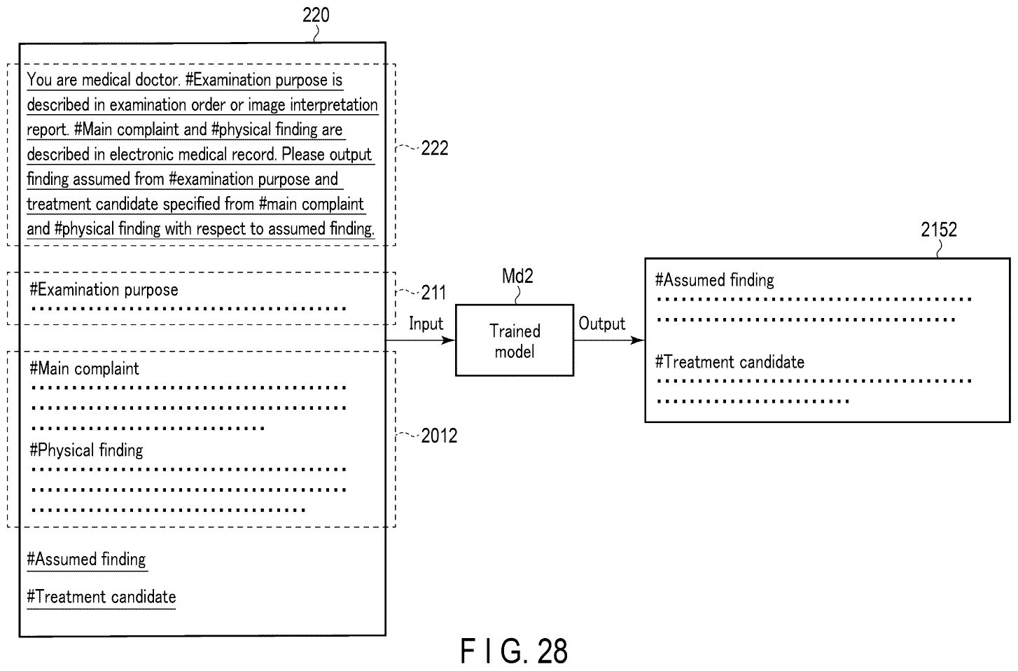

The processing circuitry 15 of the medical information processing apparatus 1 outputs the output sentence 2152 based on the instruction 222 and displays the output sentence on the display 13 in a case where the input sentence 220 including the instruction 222, the examination purpose information 211, and the description 2012 of the main complaint and the physical finding of the patient is input by the operation of the medical doctor, as illustrated in FIG. 28. That is, the output sentence 2152 includes description content for “#assumed finding” and description content for “#treatment candidate”. Note that the description content for the “#treatment candidate” is not limited to a single candidate, and may include a plurality of about three candidates.

The medical doctor understands the treatment candidates for the finding assumed from the examination purpose based on the displayed output sentence 2152. Thereafter, the medical doctor explains the finding and the candidate selected from the treatment candidates to the patient. As described above, with the configuration of displaying the finding assumed from the patient's main complaint and the physical findings, and the treatment candidates, it is possible to support explanation to the patient by the medical doctor. In addition, the processing circuitry 15 selects one of the treatment candidates included in the output sentence 2152 by the operation of the medical doctor, and inputs the selected candidate as a treatment to the electronic medical record 201, so that it is possible to reduce labor of input of the treatment.

As described above, according to the fifth embodiment, the processing circuitry 15 includes the generative AI function 15g. In a case where the instruction 222 for outputting the finding assumed from the examination purpose information 211 or the image interpretation information 212 and the treatment candidate specified from the main complaint and the physical finding of the patient with respect to the assumed finding, the examination purpose information 211 or the image interpretation information 212 corresponding to the instruction 222, and the main complaint and the physical finding are input, the processing circuitry 15 outputs and displays the assumed finding and the treatment candidate on the display 13. Therefore, it is possible to prevent overlooking of the important finding regarding the examination by confirming the important finding for the assumed finding, based on the content displayed according to the instruction 222. Moreover, it is possible to support the medical doctor for explanation to the patient by outputting treatment candidates following the patient's wish, which have been specified from the patient's main complaint and physical finding. Furthermore, it is possible to reduce labor of input of the treatment by selecting one of the treatment candidates and inputting the selected candidate as a treatment to the electronic medical record 201.

### (Modification)

In the fifth embodiment, the input sentence 220 including the #main complaint and the #physical finding described in the electronic medical record 201 is input to the trained model Md2, but the present embodiment is not limited thereto. For example, the input sentence 220 created by the medical doctor inputting the #main complaint and the #physical finding included in the content of conversation with the patient may be input to the trained model Md2. Along with this, a part of the instruction 222 may be changed to a sentence not including the electronic medical record, such as “The following sentence describes #main complaint and #physical finding based on the conversation with the patient”. As a result, even in a case where the patient's main complaint and physical finding are not in the electronic medical record 201 but in the head of the medical doctor, similar effects to those of the fifth embodiment can be obtained.

Furthermore, in the fifth embodiment, the second generative AI function 15i outputs the #assumed finding and the #treatment candidate based on the main complaint and the physical finding of the electronic medical record 201, but the present embodiment is not limited thereto. For example, the second generative AI function 15i may output the #assumed finding based on the main complaint and the physical finding of the electronic medical record 201, output the #patient's wish based on the main complaint and the physical finding of the electronic medical record 201, output the #standard treatment based on the output #assumed finding, and output the #treatment candidate based on the output #standard treatment and #patient's wish. In this case, the medical doctor may change a part of the instruction 222 according to a change in the output format. As a result, it is possible to support the explanation of the standard treatment and the patient's wish to the patient in addition to the effects of the fifth embodiment.

Further, in the fifth embodiment, one of the treatment candidates is copied and input to the electronic medical record 201, but the present embodiment is not limited thereto. For example, in a case where there is no good candidate, a treatment assumed by the medical doctor may be manually input to the electronic medical record 201.

According to at least one embodiment described above, it is possible to specify the incidental finding and give attention to the incidental finding while suppressing an increase in cost.

The term “processor” used in the above description means, for example, a CPU, a GPU, or a circuit such as an application specific integrated circuit (ASIC), a programmable logic device (for example, a simple programmable logic device (SPLD)), a complex programmable logic device (CPLD), or a field programmable gate array (FPGA). In a case where the processor is, for example, a CPU, the processor reads and executes a program stored in a memory to implement a function. Meanwhile, in a case where the processor is, for example, an ASIC, instead of storing a program in a memory, the function is directly incorporated as a logic circuit in a circuit of the processor. Note that each processor of the present embodiment is not limited to a case where each processor is configured as a single circuit, and a plurality of independent circuits may be combined and configured as one processor to implement the function. Furthermore, a plurality of components in FIG. 1, 2, 13, 16, or 27 may be integrated into one processor to realize the functions

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information processing apparatus comprising processing circuitry configured to:
acquire examination purpose information regarding an examination purpose including a description regarding a first disease candidate of a patient;
acquire image interpretation information including the description regarding the first disease candidate from an image interpretation report regarding an image examination performed according to the examination purpose;
specify an incidental finding described for a second disease candidate different from the first disease candidate from the image interpretation information; and
display a specified result on a display.

2. The medical information processing apparatus according to claim 1, wherein the processing circuitry is configured to highlight and display the specified result on the display.

3. The medical information processing apparatus according to claim 1, wherein the processing circuitry is configured to further specify the description regarding the first disease candidate from the image interpretation information.

4. The medical information processing apparatus according to claim 3, wherein the processing circuitry is configured to display the specified result on the display so as to distinguish the incidental finding and the description regarding the first disease candidate.

5. The medical information processing apparatus according to claim 4, wherein the processing circuitry is configured to display that the incidental finding is inconsistent with the examination purpose and the description regarding the first disease candidate is consistent with the examination purpose on the display.

6. The medical information processing apparatus according to claim 4, wherein the processing circuitry is configured to highlight and display the incidental finding and the description regarding the first disease candidate on the display.

7. The medical information processing apparatus according to claim 1, wherein
the processing circuitry is configured to:
extract the first disease candidate from the examination purpose information;
extract, from the image interpretation information, an image interpretation disease candidate including at least the first disease candidate out of the first disease candidate and the second disease candidate; and
specify the incidental finding based on the extracted first disease candidate and the image interpretation disease candidate.

8. The medical information processing apparatus according to claim 7, wherein
the processing circuitry is configured to:
extract the first disease candidate by analyzing the examination purpose information; and
extract the image interpretation disease candidate by analyzing the image interpretation information.

9. The medical information processing apparatus according to claim 3, wherein
the processing circuitry includes generative AI, and
in a case where an instruction for specifying the incidental finding and the description regarding the first disease candidate from the examination purpose information and the image interpretation information, the examination purpose information, and the image interpretation information are input, the generative AI outputs the specified result and displays the specified result on the display.

10. The medical information processing apparatus according to claim 9, wherein the processing circuitry is further configured to further output an importance level for the specified result based on the image interpretation information and display the importance level on the display.

11. The medical information processing apparatus according to claim 10, wherein the processing circuitry is configured to control the display to further display the importance level for the second disease candidate described in the incidental finding of the specified result based on a relationship between a predetermined disease candidate and the importance level.

12. The medical information processing apparatus according to claim 1, wherein the processing circuitry is configured to acquire the examination purpose information based on at least one of an electronic medical record of the patient, an examination order of the image examination, or the image interpretation report.

13. The medical information processing apparatus according to claim 12, wherein
the processing circuitry acquires the examination purpose information based on at least items of patient information, the examination purpose, and suspicion of diagnosis among the items of the patient information, main complaint, previous disease, concurrent disease, internal medicine, family history, physical finding, the examination purpose, and the suspicion of diagnosis regarding the patient.

14. The medical information processing apparatus according to claim 1, wherein
the processing circuitry acquires the image interpretation information based on at least an item of finding among items of the finding, evaluation and interpretation, and image interpretation result included in the image interpretation report.

15. The medical information processing apparatus according to claim 1, wherein the processing circuitry is further configured to notify a clinical department regarding the specified result of the specified result and patient information regarding the patient.

16. The medical information processing apparatus according to claim 1, wherein the processing circuitry is further configured to transmit examination reservation information of the patient to an examination department regarding the specified result.

17. The medical information processing apparatus according to claim 12, wherein
the processing circuitry includes generative AI, and
in a case where an instruction for outputting a finding assumed from the examination purpose information or the image interpretation information and whether or not treatment for the assumed finding is described in the electronic medical record, the examination purpose information or the image interpretation information corresponding to the instruction, and the electronic medical record are input, the generative AI outputs the assumed finding and whether or not the treatment is described in the electronic medical record and displays the output on the display.

18. The medical information processing apparatus according to claim 12, wherein
the processing circuitry includes generative AI, and
in a case where an instruction for outputting a finding assumed from the examination purpose information or the image interpretation information, and a treatment candidate specified from a main complaint of the patient and a physical finding for the assumed finding, the examination purpose information or the image interpretation information corresponding to the instruction, and the main complaint and the physical finding are input, the generative AI outputs the assumed finding and the treatment candidate, and displays the output on the display.

19. A medical information processing method comprising:
acquiring examination purpose information regarding an examination purpose including a description regarding a first disease candidate of a patient, and an image interpretation report regarding an image examination performed according to the examination purpose;
acquiring image interpretation information including the description regarding the first disease candidate from the image interpretation report;
specifying an incidental finding described for a second disease candidate different from the first disease candidate in the image interpretation information; and
displaying a specified result on a display.

20. A non-transitory computer readable storage medium including computer executable instructions, wherein the instructions, when executed by a processor, cause the processor to perform a method comprising:
acquiring examination purpose information regarding an examination purpose including a description regarding a first disease candidate of a patient, and an image interpretation report regarding an image examination performed according to the examination purpose;
acquiring image interpretation information including the description regarding the first disease candidate from the image interpretation report;
specifying an incidental finding described for a second disease candidate different from the first disease candidate in the image interpretation information; and
displaying a specified result on a display.
